(19) United States
(12) Patent Application Publication
(10) Pub. No.: US 2006/0177387 A1
(43) Pub. Date: Aug. 10, 2006

(54) Compositions comprising bone marrow cells, demineralized bone matrix and various site-reactive polymers for use in the induction of bone and cartilage formation

(76) Inventors: Shimon Slavin, Jerusalem (IL); Olga Gurevitch, Jerusalem (IL); Basan Gowda S Kurkalli, Nesher (IL); Daniel A Cohn, Jerusalem (IL); Aleiandro Sosnik, Jersusalem (IL)

Correspondence Address: LADAS & PARRY, 26 WEST 61ST STREET, NEW YORK, NY, 10023, US

(21) Appl. No.: 10/526,597

(22) PCT Filed: Sep. 4, 2003
(86) PCT No.: PCT/IL03/00728

§ 371 Date: Oct. 3, 2005

(51) Int. Cl. A61K 35/32

(52) U.S. Cl. 424/50; 424/93.7

(57) ABSTRACT

A composition comprising bone marrow cells (BMC) and demineralized bone matrix (DBM) or demineralized tooth matrix (DTM), together with a site-responsive polymer, optionally further comprising bone morphogenetic proteins (BMP) and/or other active agents, particularly for use in the transplantation of mesenchymal progenitor cells into a joint or a cranio-facial-maxillary bone, alveolar bone of maxilla and mandibula, spine, pelvis or long bones, or for construction or reconstruction of any extra skeletal bone, including for mechanical or biological support of artificial implants to the joint or of the joint or to the bone, for restoring and/or enhancing the formation of a new hyaline cartilage and subchondral bone structure. A kit is provided for performing transplantation of the composition into a joint, maxillary or mandibular alveolar bone or any bony structure of a mammal, including support of artificial implants.

### FIELD OF THE INVENTION

The present invention relates to compositions comprising bone marrow cells (BMC) and demineralized bone matrix (DBM), supplemented with a site-responsive polymer, and to their novel uses in induction of new bone and cartilage formation in mammals.

### BACKGROUND OF THE INVENTION

New bone formation, such as in the case of damage repair or substitution of a removed part of the bone in postnatal mammals, can only occur in the presence of the following three essential components, (i) mesenchymal progenitor cells; (ii) a conductive scaffold for these cells to infiltrate and populate; and (iii) active factors inducing chondro- and osteogenesis. In addition, for successful repair or replenishment of damaged hard tissues having definite mechanical functions, integrity and stability of the shape should be conferred to the transplant, withstanding mechanical forces during the period of tissue regeneration. Unfortunately, local conditions usually do not satisfy the requirements of osteogenesis, and thus substitution of removed, damaged or destroyed bones does not occur spontaneously.

Previous research has already uncovered somewhat about these three components.

It was shown that multipotent mesenchymal stem cells, which are capable of extensive proliferation and differentiation into cartilage, bone, tendon, muscle, fat and etc. are present in the bone marrow [Caplan, A. I. (1991) J Orthop Res 9:641-650; Prockop J. D. (1997) Science 276:71-74; Pittehger, M. F. et al. (1999) Science 284:143-147; Wakitani, S. W. et al. (1995) Muscle & Nerve 18:1417-1426].

DBM has been shown to play the role of supportive material or structure that is essential for promoting engraftment of mesenchymal progenitor cells and their proliferation and differentiation in the course of bone and cartilage development, whenever mesenchymal cells are introduced as a cell suspension (Inventors' unpublished results). It serves as a conductive scaffold for cartilage and bone regeneration, while providing a natural source for inducing both chondro- and osteogenesis, thus combining all the essential inductive and conductive features. DBM also has additional advantageous, that can be summarized as follows: (i) it is mechanically flexible and slowly biodegradable, with the degradation time compatible with the period of de novo chondro- and osteogenesis; (ii) it is strong enough to provide at least partially biomechanical properties of the flat bone and joint surface during the period of new bone and cartilage formation; (iii) it can be provided as an amorphous powder that can be inserted locally, without major surgical intervention, while avoiding iatrogenic damage; (iv) it is a low immunogenic material even when used as a xenograft, and when used in an allogeneic combination, it is practically non-immunogenic [Block, J. E. and Poser, J. (1995) Med Hypotheses 45(1):27-32; Torricelli, P. et al. (1999) Int Orthop 23(3):178-81; Hallfeldt, K. K. et al. (1995) J Surg Res 59(5):614-20].

Most importantly, DBM is also a natural source for Bone Morphogenic Proteins (BMPs)—growth factors that play an important role in the formation of bone and cartilage [Ducy, P. and Karsenty, G. (2000) Kidney Int 57(6):2207-14; Schmitt, J. M. et al. (1999) J Orthop Res 17(2):269-78]. Moreover, induction of cartilage and bone may be enhanced by additional exogenous supply of BMPs that are not even species-specific [Sampath, T. K. and Reddi, A. H. (1983) Proc Natl Acad Sci USA 80(21): 6591-5; Bessho, K. et al. (1992) J Oral Maxillofac Surg 50(5):496-501], together with DBM [Niederwanger, M. and Urist, M. R. (1996) J Oral Implantol 22(3-4):210-5].

Arthropathies are a group of chronic progressive joint diseases that can result from degenerative changes in the cartilage and hypertrophy of bone at the articular margins. Arthropathies can be secondary to trauma, inflammatory (autoimmune or infectious), metabolic or neurogenic diseases. Hereditary and mechanical factors may be an additional factor involved in the pathogenesis of arthropathies.

Restoration of a healthy joint surface in a damaged or degenerative arthropathy requires addressing the treatment both towards the cartilage and the subchondral bone.

Various attempts have been made to replace damaged cartilage, including:

1. Stimulation of bone marrow from subchondral bone to form a fibrotic repair tissue;

2. Osteochondral transplantation (allogeneic and autologous);

3. Transplantation of autologous cultured chondrocytes or mesenchymal cells;

4. Combined transplantation of chondrocytes with different kinds of matrices; and

5. Artificial implantation of mechanical joints.

Each of these methods has limitations and disadvantages and most of them are expensive, cumbersome, ineffective and rather impractical. Autologous osteochondral graft is restricted to a small area of damaged cartilage, up to 2 cm², and could cause discomfort, infection and morbidity in the donor site. Allogeneic osteochondral graft is immunogenic, hence requires life-long use of undesired, hazardous immunosuppressive agents, which would be an impractical approach for routine orthopedic practice. Transplantation of cultured chondrocytes is cumbersome and very expensive, involving a two-stage procedure. The hyaline-like tissue which is produced after transplantation has sub-optimal biomechanical properties [Gilbert, J. E. (1998) Am J Knee Surg 11(1):42-6; Temeno, J. S. and Mikos, A. G. (2000) Biomaterials: Tissue Engineering for Regeneration of Articular Cartilage, 21:431-440; Buckwalter, J. A. and Mankin, H. J. (1998) Instr Course Lect 47:487-504; Stocum, D. L. (1998) Wound Repair Regen. 6(4):276-90]. Hence, adequate restoration of cartilage remains an unsolved problem.

Currently, autologous grafts are the most commonly used bone and cartilage graft material. However, the use of autografts has limitations, such as donor site discomfort, infection and morbidity and limited sizes and shapes of available grafts. Even if enough tissue is transplanted there is an acute limitation in the number of mesenchymal stem cells with high proliferative potential present in the differentiated bone tissue implanted.

The most promising approach should involve the combined transplantation of cells capable of formation of both hyaline cartilage and subchondral bone and a matrix, providing means for induction/conduction and support of bone and cartilage development and maintenance.

It is widely accepted that, for successful application of combined cell-matrix graft, the basic requirements are the following:

1. Rich source of progenitor cells capable of differentiation into chondrocytes, for continuous repair of “wear and tear” of weight bearing joints.

2. Conductive scaffold for cell attachment should be maintained, leading to development of hyaline cartilage.

3. Conductive scaffold should be non-immunogenic, non-toxic and susceptible to biodegradation simultaneously with the development of new cartilage.

4. Conditions for stimulating development of chondrocytes from mesenchymal precursor cells.

So far, most of the matrices that were tried in combined cell-matrix grafts were either immunogenic or non-biodegradable, and the remaining others did not possess conductive or inductive properties needed to support formation of biomechanical strong cartilage. Cells used in combined cell-matrix grafts were in most of the cases chondrocytes, which were already fully differentiated cells, with relatively low metabolic activity and limited self-renewal capacity. Whereas the proliferative capacity of such cells may be sufficient to maintain healthy cartilage, it is certainly insufficient for the development de novo of large areas of hyaline cartilage. In addition to being immunogenic, mesenchymal progenitor cell allografts were not combined with optimal supportive matrix. Thus, unfortunately, none of the available options fulfill all basic requirements, and all options are far from being satisfactory for reliable routine clinical application.

WO02/070023, fully incorporated herein by reference, describes a composition comprising BMC and DBM and/or MBM which provides, upon administration into a damaged joint, replacement and/or restoration of hyaline cartilage together with subchondral bone, in a one-step transplantation procedure, without any preliminary cultivation of mesenchymal progenitor cells. As shown in PCT/IL02/00172, the application of the two components, BMC and DBM together, is both essential and sufficient for the development of new bone and cartilage at the place of the transplantation. This method can be successfully used to initiate and/or improve the efficiency of bone and cartilage formation. It proved to be very effective in (i) repair of damaged osteochondral complex in joints; and (ii) repair or replenishment of the bones in cranio-maxillo-facial areas (for therapeutic and cosmetic purposes). The composition induced the development of the new bone and cartilage according to the local conditions of the site of transplantation. New tissue formation follows a differentiation pathway producing different types of bone and cartilage, depending on the local conditions. Thus, the newly formed tissue meets precisely the local demands.

A major prerequisite for successful replenishment of damaged bone and cartilage structures by transplantation of BMC-DBM composition is the ability to provide for the integrity and stability of shape of the transplant, withstanding mechanical influence during the period of tissue regeneration, whilst maintaining ease of administration.

The administration into a damaged joint or bone of a syringeable usually relatively non-viscous composition, may therefore require keeping the patient at rest and in an unchanging position, until adequate induction of bone and/or cartilage is initiated and obtained, to prevent the composition from migrating and leaving the injection site. In order to improve the procedure, the present inventors developed an improved composition, which comprises in addition to BMC and DBM, a site-responsive polymer, which is liquid (and thus syringeable) at ambient temperature, and gels at body temperature. This composition, which is a major object of the present invention, forms a stable depot at the site of injection, which enables the maintenance of the integrity and stability of shape of the transplant, whilst providing mechanical properties essential to temporarily meet the requirements of the recipient throughout the period of tissue regeneration.

The goals of supplementing the active bone and cartilage regenerating complex consisting of DBM and BMC with additional polymeric materials are therefore:

1. To maintain integrity and shape of the transplanted complex.

2. To provide the transplanted complex with the mechanical properties essential to temporarily meet the requirements of the organism (such as withstanding physical and mechanical pressure, etc.) throughout the period of tissue regeneration.

The inventors have interestingly found that the addition of biocompatible, site-responsive polymers achieves these objects.

Thus, in order to improve the properties of a BMC-DBM active complex, it may be supplemented with a substance possessing the following features:

1. The supplement has to be compatible with proliferation and differentiation of mesenchymal progenitor cells, in the course of bone and/or cartilage formation.

2. The supplement has to be slowly biodegradable or dissolvable in the body fluids, the degradation time being compatible with the period of de novo chondro- and osteogenesis.

3. The supplement has to be non-immunogenic.

4. The supplement has to be provided in a form (state) allowing its mixing with the components of the active complex (DBM and BMC).

5. The supplement after its admixture with the active complex has to render it sufficiently strong to maintain integrity and shape as well as to provide biomechanical properties to the transplant during the period of new tissue formation.

The inventors have now found that when using a site-responsive polymer, which may be either a reverse thermogelating polymer (RTG), or a modified polymer containing reactive Si-based moieties capable of generating stable and inert Si—O—Si bonds in the presence of water as the supplement, these requirements are met, leading to very impressive results as described in the following Examples.

Biomaterials are materials which are foreign to the human body and can be used in direct contact with its organs, tissues and fluids. These materials include, among others, polymers, ceramics, biological materials, metals, composite materials and combinations thereof. A major prerequisite of polymeric biomaterials is their syringeability, namely being suitable to be implanted without requiring a surgical procedure. Injectable polymers combine low viscosity at the injection stage (at room temperature), with a gel or solid consistency developed in situ, later on (at body temperature). The syringeability of injectable biopolymers is their most essential advantage, since it allows their introduction into the body using minimally invasive techniques. Furthermore, their low viscosity and substantial flowability at the injection time, enable them to reach and fill spaces, otherwise inaccessible, as well as to achieve enhanced attachment and improved conformability to the tissues at the implantation site. On the other hand, a sharp increase in viscosity is a fundamental requirement for these materials to be able to fulfill any physical or mechanical function. The high viscosities play a critical role also in that the generating syringeable materials, once at the implantation site, can control the rate of release of drugs, or can function as the matrix for cell growth and tissue scaffolding. Clearly, biodegradability is yet another important requirement for some of these materials.

U.S. Pat. No. 5,939,485 discloses responsive polymer networks exhibiting the property of reversible gelation triggered by a change in diverse environmental stimuli, such as temperature, pH and ionic strength. U.S. Pat. No. 6,201,065 discloses thermo-responsive macromers based on cross-linkable polyols, such as PEO-PPO-PEO triblocks, capable of gelling in an aqueous solution, which can be covalently crosslinked to form a gel on a tissue surface in vivo. The gels are useful in a variety of medical applications including drug delivery.

The term “thermo-sensitive” refers to the capability of a polymeric system to achieve significant chemical, mechanical or physical changes due to small temperature differentials. In order to avoid open surgical procedure, thermo-responsive materials have to be easily syringeable, combining low viscosity at the injection stage, with a gel or solid consistency developed later on, in situ.

The reverse thermo-responsive phenomenon is usually known as Reverse Thermal Gelation (RTG). Water solutions of RTG materials display low viscosity at ambient temperature, and exhibit a sharp viscosity increase as temperature rises within a very narrow temperature range, producing a semi-solid gel once they reach body temperature. There are known several RTG displaying polymers, such as poly(N-isopropyl acrylamide) (PNIPAAm) (e.g. U.S. Pat. No. 5,403,893). Unfortunately, N-isopropylacrylamide is toxic, and moreover poly(N-isopropyl acrylamide) is non-degradable and, in consequence, is not suitable where biodegradability is required.

One of the most important RTG-displaying materials is the family of poly(ethylene oxide)/poly(propylene oxide)/poly(ethylene oxide) (PEO-PPO-PEO) triblocks, available commercially as Pluronic® (U.S. Pat. No. 4,188,373). By adjusting the concentration of the polymer, the desired liquid-gel transition can be obtained, nevertheless, relatively high concentrations of the triblock (typically above 15-20%) are required. Another known system which is liquid at room temperature, and becomes a semi-solid when warmed to about body temperature, is disclosed in U.S. Pat. No. 5,252,318, and consists of tetrafunctional block polymers of polyoxyethylene and polyoxypropylene condensed with ethylenediamine (commercially available as Tetronic®).

However, for most known RTG polymers, even though they exhibit a significant increase in viscosity when heated up to 37° C., the levels of viscosity attained are not high enough for most clinical applications. Due to this fundamental limitation, these systems display unsatisfactory mechanical properties and unacceptable short residence times at the implantation/injection site. Furthermore, due to these characteristics, these gels have high permeability, a property which renders them unsuitable for drug delivery applications because of the fast drug release kinetics of these gels. Despite their clinical potential, these materials have failed to be used successfully in the clinic, because of serious performance limitations [Steinleitner et al., Obstetrics and Gynecology, 77, 48 (1991); Esposito et al., Int. J. Pharm. 142, 9 (1996)].

Biodegradability plays a unique role in a diversity of devices, implants and prostheses. Biodegradable polymers need not be removed from the body and can serve as matrices for the release of bioactive molecules and result in improved healing and tissue regeneration processes. Biodegradable polymers such as polyesters of α-hydroxy acids, like lactic acid or glycolic acid, are used in diverse applications such as bioabsorbable surgical sutures and staples, some orthopedic and dental devices, drug delivery systems and more advanced applications such as the absorbable component of selectively biodegradable vascular grafts, or as temporary scaffold for tissue engineering. Biodegradable polyanhydrides and polyorthoesters, having labile backbone linkages, have also been developed. Polymers which degrade into naturally occurring materials, such as polyaminoacids, have also been synthesized. Degradable polymers formed by copolymerization of lactide, glycolide, and ε-caprolactone have been disclosed. Polyester-ethers have been produced by copolymerizing lactide, glycolide or α-caprolactone with polyethers, such as polyethylene glycol (“PEG”), to increase the hydrophilicity and degradation rate.

Unfortunately, the few absorbable polymers clinically available today are stiff, hydrophobic solids, therefore clearly unsuitable for non- or minimally invasive surgical procedures, where injectability is a fundamental requirement. The only way to avoid the surgical procedure with these polymers, is to inject them as micro or nanoparticles or capsules, typically containing a drug to be released. As an example, injectable implants comprising calcium phosphate particles in aqueous viscous polymeric gels, were first proposed in US 5,204,382. Even though the ceramic component in these polymers is generally considered to be nontoxic, the use of nonabsorbable particulate material seems to trigger a foreign body response both at the site of implantation as well as at remote sites, due to the migration of the particles, over time.

Another approach is the in situ precipitation technique described in U.S. Pat. No. 4,938,763, where a water soluble organic solvent is used, in which the polymer is soluble. Once the system is injected, the organic solvent gradually dissolves in the aqueous biological medium, leaving behind an increasingly concentrated polymer solution, until the polymer precipitates, generating the solid implant in situ.

In situ polymerization and/or crosslinking are another important techniques used to generate injectable polymeric systems. For example, U.S. Pat. No. 5,410,016 describes water soluble low molecular precursors having at least two polymerizable groups, that are syringed into the site and then polymerized and/or crosslinked in situ chemically or preferably by exposing the system to UV or visible radiation. Langer et al. [Biomaterials, 21, 259-265 (2000)] developed injectable polymeric systems based on the percutaneous polymerization of precursors, using UV radiation. An additional approach was disclosed in U.S. Pat. No. 5,824,333 based on the injection of hydrophobic bioabsorbable liquid copolymers, suitable for use in soft tissue repair.

Although known RTG polymers like Pluronic® may be used as supplements for the BMC-DBM complex used in the present invention, the inventors have also developed novel RTG polymers, which overcome many of the drawbacks of prior art polymers and techniques. The use of these novel polymers in bone and cartilage induction and rehabilitation is also an object of this invention. These polymers will be described in detail hereafter. In addition to RTG polymers, the inventors developed a responsive “polymeric system”, which is an organic-inorganic environmentally, or site responsive polymeric system. This system, described in detail hereafter, is characterized by having at least one polymeric component which comprises silicon-containing reactive groups, and can be RTG and/or otherwise responsive to environmental triggers. Compositions comprising this polymeric system are also an object of the present invention.

The sol-gel process whereby inorganic networks are formed from silicon or metal allioxide monomer precursors is broadly used in diverse areas, including the glass and ceramic fields. Typically, three reactions are involved in the sol-gel process, namely hydrolysis, alcohol condensation, and water condensation. One of the main advantages of this method, is that homogeneous inorganic oxide materials with valuable properties such as chemical durability, hardness, optical transparency, appropriate porosity and thermal resistance, can be produced at room temperature. This, as opposed to the much higher temperatures required in the production of conventional inorganic glasses.

The most widely used materials are alkoxysilanes such as tetramethoxysilane (TMOS) and tetraethoxysilane (TEOS). A number of factors will significantly affect the characteristics and properties of a particular sol-gel inorganic network. Especially important are temperature and pH, on one hand, and the type and concentration of the catalyst and the water/silicon molar ratio.

The hydrolysis of the alkoxide groups (OR) results in their replacement with hydroxyl moieties (OH). The subsequent condensation reaction involving the silanol groups (Si—OH) produces siloxane bonds (Si—O-Si) plus the by-products water or alcohol. The relative rate of the hydrolysis and condensation reactions is such that, under most conditions, the latter starts before the former is complete. By fine tuning various experimental parameters such as the pH of the system, the H₂O/Si molar ratio and the type of catalyst, the hydrolysis reaction can be brought to completion before the condensation step starts.

The polymerization process can be conducted in three different pH regions: below pH=2, between pH=2 and pH=7 and for pH values higher than 7. The overall process occurs in three stages: (i) First, particles form due to the polymerization of the precursors; (ii) Then, the particles grow and finally (iii) The particles join forming chains and then networks that extend throughout the liquid medium, thickening into a gel.

It has been observed that the rate and extent of the hydrolysis reaction is largely influenced by both the strength and the concentration of the acid or base catalyst. It has been reported that the reaction is faster for pH values below 5 or, alternatively, above 7 [Bourges X. et al., Biopolymers, 63, 232 (2002)]. Expectedly, larger H₂O/Si molar ratios normally encourage hydrolysis. It should be also stressed that, since water is the by-product of the condensation reaction, large water contents promote siloxane bond hydrolysis.

The pH of the medium plays an important role also during the condensation stage. At pH values between 6 and 7 the reaction is at its lowest pace, while in the 2-6 pH range and above pH=7 the reaction is similarly faster. In addition, even though the condensation stage can proceed without catalyst, their use is helpful. The acid-catalyzed condensation mechanism involves the protonation of the silanol species, as a result of which the silicon becomes more electrophilic and, thus, more susceptible to nucleophilic attack. The most widely accepted mechanism for the base-catalyzed condensation reaction involves the attack of a nucleophilic deprotonated silanol on a neutral silicic acid.

As it pertains to the structure of the materials obtained, it can be stated that when the reaction is performed under acidic conditions, the sol-gel derived silicon oxide networks primarily comprise linear or randomly branched polymers which, in turn, entangle and form additional branches resulting in gelation. On the other hand, silicon oxide networks obtained under base-catalyzed conditions produce more highly branched clusters which do not interpenetrate prior to gelation and thus behave as discrete clusters.

Some work has been conducted aiming. at developing inorganic-organic telechelic polymers. For example, Bunel et al [Polymer, 39, 965 and 973 (1998)] described the functionalization of low molecular weight polybutadiene chains with triethoxysilane and their crosslinking at temperatures ranging from 20° C. to 80° C. for 30 days. Seppala and co-workers [Polymer, 42, 3345 (2001)] reported the modification of polylactic acid. The crosslinking was carried out at drastic conditions: 60° C.-120° C. in presence of nitric acid as the catalyst. Osaka and collaborators [J. Sol-Gel Sci. Tech., 21, 115 (2001)] prepared hybrid materials incorporating gelatin and 3-(glycidoxypropyl) trimethoxysilane through sol-gel processing. Zhu and co-workers [J. Mat. Sci. Mat. Med., 14, 27 (2003)] prepared silica-butyrylchitosan hybrid films, using butyrylchitosan as the organic species incorporated into the system. The sol-gel process was carried out in hydrochloric acid and methanol at RT for several days and heating at 80° C. for 2 hours.

The use of silica to induce the formation and deposition of calcium phosphate (CaP) derivatives such as apatite and hydroxyapatite for bone regeneration, was studied. Thus, Li and collaborators [J. Biomed. Mat. Res., 29, 325 (1995)] reported that silica gels sintered at 900-1000° C. can stimulate apatite crystallization from metastable calcium phosphate solutions on their surfaces. Varma and co-workers [J. Mat. Sci. Mat. Med., 12, 767 (2001)] functionalized cotton fibers with tetraethoxysilane and studied the growth of CaP on it. Lopatin et al [J. Mat. Sci. Mat. Med., 12, 767 (2001)] used silicon substrates to grow HA and tricalcium phosphate. Finally, Nakamura and his group [Biomaterials, 24, 1349 (2003)] developed calcium oxide-containing glasses and evaluated the apatite formation in contact with simulated body fluid solution.

The presently proposed use of polymers which are responsive to an environmental trigger, e.g. RTG polymers which are responsive to body temperature, or polymers which become more viscous in response to other environmental trigger/s, would afford substantial advantages, particularly in the field of orthopedics and joint repair.

Thus, it is the major object of the present invention to provide a mixture of bone marrow cells and demineralized bone matrix, together with a site-responsive polymer, for use as a graft in patients in need of restoration of, inter alia, damaged joints and/or cranio-facial-maxillary bones, in a one-step transplantation procedure. This and other objects of the invention will be elaborated on as the description proceeds.

### SUMMARY OF THE INVENTION

The present invention relates to compositions comprising a mixture of bone marrow cells (BMC) and demineralized bone or tooth matrix (DBM or DTM, respectively), together with a site-responsive polymer and to their novel uses in the transplantation of mesenchymal progenitor cells into joints and cranio-facial-maxillary area (when the bone is absent to induce bone formation).

Thus, in a first aspect, the present invention relates to a composition comprising bone marrow cells (BMC) and demineralized bone matrix (DBM), together with a site-responsive polymer.

In a second aspect, said composition comprising BMC and DBM together with a site-responsive polymer, is intended for use in transplantation of mesenchymal progenitor cells present in the bone marrow into a joint or a cranio-facial-maxillary area of a subject in need, wherein said subject is a mammal, preferably a human.

In a first embodiment, the DBM comprised within the composition of the invention is of vertebrate origin, and may be of human origin.

In a second embodiment, the DBM comprised within the composition of the invention is in powder or particle form. The particle size of the DBM may be about 50 to 2500μ. Preferably, said particle size is about 250 to 500μ. The most preferable particle size will depend on the specific needs of each case. Alternatively, the DBM may be in string form, particularly for reconstruction of tendons, or in or larger particles of DBM or slice form for reconstruction of large bone area. Slices or large particles may be perforated, to allow for better impregnation with mesenchymal stem cells.

In another embodiment, the composition of the invention is for restoring and/or enhancing the formation of a new hyaline cartilage and/or sub-chondral bone structure.

In a further embodiment, the composition of the invention is intended for the treatment of a patient suffering from any one of a hereditary or acquired bone disorder, a hereditary or acquired cartilage disorder, a malignant bone or cartilage disorder, conditions involving bone or cartilage deformities and Paget's disease. Additionally, the invention is also intended for the treatment of a patient in need of any one of correction of complex fractures, bone replacement and formation of new bone in plastic or sexual surgery.

In a yet further embodiment, the composition of the invention may further optionally comprise a pharmaceutically acceptable carrier or diluent, as well as additional active agents.

In another aspect, the present invention relates to a method for transplantation of a mixture comprising BMC with DBM and a site-responsive polymer, optionally further comprising pharmaceutically acceptable carrier or diluent, into a joint and/or a cranio-facial-maxillary bone area of a subject in need, wherein said method comprises introducing into said joint or bone the composition of the invention.

In a first embodiment of the method of the invention, the mixture is administered by any one of the following procedures injection, minimally invasive arthroscopic procedure, or by surgical arthroplasty into the site of implantation, wherein said method is for support or correction of congenital or acquired abnormalities of the joints, cranio-facial-maxillary bones, orthodontic procedures, bone or articular bone replacement following surgery, trauma or other congenital or acquired abnormalities, and for supporting other musculoskeletal implants, particularly artificial and synthetic implants.

Thus, in a further aspect, the invention relates to a method of treating a damaged or degenerative arthropathy associated with malformation and/or dysfunction of cartilage and/or subchondral bone in a mammal in need of such treatment, comprising administering into an affected joint or bone of said mammal a mixture comprising BMC with DBM, together with a site-responsive polymer, said mixture optionally further comprising a pharmaceutically acceptable carrier or diluent and/or additional active agents.

In one embodiment, the BMC which are present in the administered mixture are either allogeneic or said mammals own.

In another embodiment, the DBM present in the administered mixture is in a powder, gel, semi-solid or solid form embedded in or encapsulated in polymeric or biodegradable materials.

In a yet further aspect, the present invention relates to a non-invasive (through injection), minimally invasive (through arthroscopy) or surgical transplantation method for support of implants of joints or other musculoskeletal implants, comprising introducing a graft into a joint or a cranio-facial-maxillary bone area of a subject in need, wherein said graft comprises a mixture of BMC and DBM, together with a SRTG polymer.

In an even further aspect, the present invention relates to the use of a composition comprising BMC and DBM, together with a polymer, as a graft of mesenchymal and/or mesenchymal progenitor cells for transplantation/implantation into a mammal, wherein said mammal is preferably a human. The transplantation is to be performed into a joint or into a cranio-facial-maxillary bone area, for the development of new bone and/or cartilage.

Furthermore, the composition used in said transplantation is intended for the treatment of a patient suffering from any one of a hereditary or acquired bone disorder, a hereditary or acquired cartilage disorder, a malignant bone or cartilage disorder, conditions involving bone or cartilage deformities and Paget's disease. In addition, said composition is intended for the treatment of a patient in need of any one of correction of complex fractures, bone replacement and formation of new bone in plastic or sexual surgery.

In one embodiment, the composition used in the invention further comprises an additional active agent.

In another embodiment, the DBM comprised within the composition of the invention is of vertebrate origin, and may be of human origin, and is preferably in powder form.

In an additional aspect, the present invention concerns the use of a mixture of BMC with DBM, together with a polymer in the preparation of a graft for the treatment of a bone or cartilage disorder.

Lastly, the present invention provides a kit for performing transplantation into a joint or for reconstruction of cranio-facial-maxillary bone area, long bones, pelvis, spines or for dental support through alveolar bone of maxilla and mandibula augmentation or for creation of an artificial hematopoietic bone of a mammal of BMC in admixture with DBM and a site-responsive polymer, wherein said kit comprises:
(a) DBM in powder or a compacted form (e.g. strings for reconstruction of tendons, or larger particles of DBM for reconstruction of large bone area);
(b) a site-responsive polymer;
(c) a BM aspiration needle;
(d) an intra-osseous bone drilling burr;
(e) a needle with a thick lumen for infusion of viscous bone marrow-DBM—site-responsive polymer mixture;
(f) a 2-way lumen connector for simultaneous mixing of BMC with DBM and site-responsive polymer and diluent;
(g) a medium for maintaining BMC; and optionally
(h) additional factors stimulating osteogenesis
(i) cryogenic means for handling and maintaining BMC or BMC together with DBM.

It is to be understood that the site-responsive polymer solution comprised in the kit of the invention may be an RTG or otherwise responsive polymer, adjusted to undergo the desired change in viscosity at the site of administration and formation of a depot in situ.

The kit of the invention may optionally further comprise a carrier and/or a diluent for the BMC and DBM and site-responsive polymer mixture.

Although the compositions of the invention may employ any suitable site-responsive polymer, like Pluronic F127, F108, etc. which are known polymers, some such polymers are preferred. Particularly preferred are novel polymers which are the subject of Israel Application No. 151588 (filed on Aug. 15, 2002), entitled Novel Thermosensitive Block Copolymers for Non-Invasive Surgery and in a publication by the present inventors [Cohn D. and Sosnik A., J. Mat. Sci. Mater. Med. 2003; 14:175-180], the contents of which are fully incorporated herein by reference. These specifically designed biodegradable reverse thermo-responsive polymers are advantageous for implantation into the human body, specifically for providing a temporary scaffold for tissue repair, and overcome many of the drawbacks of prior art polymers. These polymers covalently combine hydrophobic and hydrophilic segments. The balance between such segments in the molecule plays a dominant role in achieving the desired reverse thermal gelation (RTG) behavior.

The most preferred compositions of the present invention are tailor-made, by capitalizing on the uniqueness of the Reverse Thermal Gelation phenomenon. The endothermic phase transition taking place, is driven by the entropy gained due to the release of water molecules bound to the hydrophobic groups in the polymer backbone. It is clear, therefore, that, in addition to molecular weight considerations and chain mobility parameters, the balance between hydrophilic and hydrophobic moieties in the molecule, plays a crucial role. Consequently, the properties of different materials were adjusted and balanced by variations of the basic chemistry, composition and molecular weight of the different components.

More specifically, in one of the preferred embodiments, the RTG polymers applicable in the compositions and methods of the present invention are selected from the group consisting of polymers having the general formulae:

(a) [-Xₙ-A-Xₙ-E-B-E-]ₘ defined herein as formula Ia;

(b) [-Xₙ-B-Xₙ-E-A-E-]ₘ defined herein as formula Ib;

(c) M-Xₙ-E-B-E-Xₙ-M defined herein as formula Ia;

(d) N-Xₙ-E-A-E-Xₙ-N defined herein as formula IIb;

(e) [-Xₙ-A(Xₙ)_{y}(E)_{y}(B)_{y}-Xₙ-E-B-E-]ₘ defined herein as formula IIIa;

(f) [-Xₙ-B(Xₙ)_{y}(E)_{y}(A)_{y}-Xₙ-E-A-E-]ₘ defined herein as formula IIIB;

wherein A represents a bifunctional, trifunctional or multifunctional hydrophilic segment; M represents a monofunctional hydrophilic segment; B represents a bifunctional, trifunctional or multifunctional hydrophobic segment; N represents a monofunctional hydrophobic segment; X represents a bifunctional degradable segment; E represents bi, tri or multifunctional chain extender or coupler; n and m represent the respective degree of polymerization and y designates the additional functionality of the corresponding segment (wherein y>2).

In a particularly preferred embodiment, A is presented by polyoxyethylene or polyethylene glycol (PEG) units (O—CH₂—CH₂)_{y} [y represents degree of polymerization] carrying functional groups such as —OH, —SH, —COOH, —NH₂, —CN or —NCO groups. Consequently, A may represent poly(oxyethylene triol), poly(oxyethylene triamine), poly(oxyethylene triacarboxylic acid), ethoxylated trimethylolpropane, or any other multifunctional hydrophilic segment.

In a particularly preferred embodiment, B is presented by polyoxyalkylene (wherein the alkylene containing more than two C atoms), such as, for example, poly(propylene glycol) (PPG) units [—O—CH(CH₃)—CH₂]_{y} [wherein y represents degree of polymerization] carrying functional groups such as —OH, —SH, —COOH, —NH₂, —CN or —NCO groups. Consequently, B may represents polyoxypropylene diamine (Jeffamine.®), polytetramethylene glycol (PTMG), polyesters selected from the group consisting of poly(caprolactone), poly(lactic acid), poly(glycolic acid) or combinations or copolymers thereof, polyamides or polyanhydrides or any other bifunctional hydrophobic segment having the appropriate functional group. Trifunctional hydrophobic segment may be selected from the group consisting of poly(oxypropylene triol), poly(oxypropylene triamine), poly(oxypropylene triacarboxylic acid), or any other trifunctional hydrophobic segment.

E is preferably a chain extender or coupling segment derived from a bifunctional reactive molecule, preferably selected from the group consisting of phosgene, aliphatic or aromatic dicarboxylic acids or their reactive derivatives, such as oxalyl chloride, malonyl chloride, succinyl chloride, glutaryl chloride, fumaryl chloride, adipoyl chloride, suberoyl chloride, pimeloyl chloride, sebacoyl chloride, terephtlialoyl chloride, isophthaloyl chloride, phthaloyl chloride and/or mixtures thereof. E may be further presented by amino acids, such as for example, glycine, alanine, valine, phenylalanine, leucine, isoleucine etc.; oligopeptides, such as RGD (Arg-Gly-Asp), RGD(S) (Arg-Gly-Asp(-Ser)), aliphatic or aromatic diamines such as, for example, ethylene diamine, propylene diamine, butylene diamine, etc.; aliphatic or aromatic diols, such as ethylene diol, propanediol, butylenediol, etc.; aliphatic or aromatic diisocyanates, for example, hexamethylene diisocyanate, methylene bisphenyldiisocyanate, methylene biscyclohexane-diisocyanate, tolylene diisocyanate or isophorone diisocyanate. Trifunctional reactive molecules may be cyanuric chloride, triisocyanates, triamines, triols, trifunctional aminoacids, such as lysine, serine, threonine, methionine, asparagine, glutamate, glutamine, histidine, or oligopeptides. E may also comprise combinations of the functional groups described above in the same molecule. The reaction products are poly(ether-carbonate)s, poly(ether-ester)s, poly(ether-urethane)s or derivatives of chlorotriazine, most preferably poly(ether-carbonate)s, poly(ether-ester)s or poly(ether-urethanes), poly-imides, polyureas and combinations thereof.

In a preferred embodiment, M is presented by a monomethyl ether of hydrophilic polyoxyethylene or polyethylene glycol (PEG) units (O—CH₂—CH₂)_{y}-OCH₃ [wherein y represents a degree of polymerization] carrying functional groups such as —OH, —SH, —COOH, —NH₂, —CN or —NCO groups.

In a preferred embodiment N is presented by a monomethyl ether of hydrophobic polypropylene glycol) (PPG) units [—O—CH(CH₃)—CH₂]_{y}—OCH₃ [wherein y represents degree of polymerization] carrying functional groups such as —OH, —SH, —COOH, —NH₂, —CN or —NCO groups.

Preferred biodegradable X segments in the RTG polymers applicable in the compositions and methods of present invention possess hydrolytic instability and they are characterized by being aliphatic or aromatic esters, amides and their anhydride derivatives formed from alpha-hydroxy carboxylic acid units or their respective lactones.

According to the present invention, the most preferred RTG polymers to be employed comprise amphiphiles obtained by the combination of both hydrophobic and hydrophilic basic segments, which, separately, do not display any kind of clinically relevant viscosity change of their own, and are capable of undergoing a transition that results in a sharp increase in viscosity in response to a triggering effected at a predetermined body site and an aqueous-based solvent wherein the viscosity of said polymeric component increases by at least about 2 times upon exposure to a predetermined trigger.

More specifically, the most preferred polymers used in this invention are capable of undergoing a transition that results in a sharp increase in viscosity in response to a change in temperature at a predetermined body site; wherein the polymeric component comprises hydrophilic and hydrophobic segments covalently bound within said polymer component, by at least one chain extender or coupling agent, having at least 2 functional groups; wherein the hydrophilic and hydrophobic segments do not display Reverse Thermal Gelation behavior of their own at clinically relevant temperatures and; wherein the viscosity of said polymeric component increases by at least about 2 times upon exposure to a predetermined trigger.

In further preferred embodiments of the present invention said responsive component is a segmented block copolymer comprising polyethylene oxide (PEO) and polypropylene oxide (PPO) chains, wherein said PEO and PPO chains are connected via a chain extender, wherein said chain extender is a bifunctional, trifunctional or multifunctional molecule selected from a group consisting of phosgene, aliphatic or aromatic dicarboxylic acids, their reactive derivatives such as acyl chlorides and anhydrides, diamines, diols, aminoacids, oligopeptides, polypeptides, or cyanuric chloride or any other bifunctional, trifunctional or multifunctional coupling agent, or other molecules, synthetic or of biological origin, able to react with the mono, bi, tri or multifunctional —OH, —SH, —COOH, —NH₂, —CN or —NCO group terminated hydrophobic and hydrophilic components or any other bifunctional or multifunctional segment, and/or combinations thereof.

### BRIEF DESCRIPTION OF THE FIGURES

FIGS. 1A to 1R: Photomicrographs of mice kidney sections after subcapsular transplantation of demineralized tooth matrix and bone marrow cells with or without different polymers.

FIGS. 1A, 1B, 1C, 1D, IE & 1F: One month post-transplantation of BMC+DBM together with RTG polymers N2 (FIGS. 1A, 1B) N4 (FIGS. 1C, 1D) and N7 (FIGS. 1E, 1F) newly formed cortical and trabecular bone, well developed marrow cavity and functionally active bone marrow are seen.

FIGS. 1G & 1H: One month post-transplantation of DBM-BMC complex without RTG polymers. No difference in the developmental level of the ectopic ossicles produced by DBM-BMC complex transplanted (FIGS. 1A-1F) with or without (FIGS. 1G, 1H)) RTG polymers could be observed.

FIGS. 1I & 1J: BMC transplanted without DBM but supplemented with one of the mentioned RTG polymers produced in most of the cases a small ossicles. It means that RTG polymers successfully keep transplanted BMC together and prevent their migration out of the transplantation site.

Implantation of mentioned RTG polymers alone under the kidney capsule never left any trace in the site of transplantation—neither bone formation nor any side effects such as inflammation etc.

FIGS. 1K-1N show the use of the DTM-BMC complex in combination with the silane-based site-responsive polymers described in the invention in mice.

FIGS. 1O-1R show the use of the DBM-BMC complex in combination with the silane-based site-responsive polymers described in the invention in rats.

FIG. 1K: DTM+BMC

FIG. 1L: DTM+BMC+silane-based site-responsive polymer—Bio-polymer#21, modified Pluronic F-127 15% in water.

FIG. 1M: DTM+BMC+silane-based site-responsive polymer—Bio-polymer#22, modified Pluronic F-127 17% in water.

FIG. 1N: DTM+BMC+silane-based site-responsive polymer—Bio-polymer#23, modified Pluronic F-127 20% in water.

FIG. 1O: DBM+BMC

FIG. 1P: DBM+BMC+silane-based site-responsive polymer—Bio-polymer#21, modified Pluronic F-127 15% in water.

FIG. 1Q: DBM+BMC+silane-based site-responsive polymer—Bio-polymer#22, modified Pluronic F-127 17% in water.

FIG. 1R: DBM+BMC+silane-based site-responsive polymer—Bio-polymer#23, modified Pluronic F-127 20% in water.

One month post-transplantation of BMC+DBM (in case of rats) or BMC+DTM (in case of mice) together with silane-based site-responsive polymers (NN 21, 22 and 23) newly formed cortical and trabecular bone, well developed marrow cavity and functionally active bone marrow are seen. No difference in the developmental level of the ectopic ossicles produced by DTM-BMC complex transplanted with or without silane-based site-responsive polymers could be observed.

FIGS. 2A to 2H: Photomacro- and micrographs illustrating the experimental models of artificially created defects in osteochondral complex of knee joint and parietal region of calvarium in rats.

FIG. 2A shows a typical knee joint, FIG. 2B shows the osteochondral complex, and FIG. 2C shows normal cartilage. A standard artificial damage (experimentally created microfracture drilling) in the articular cartilage and subchondral bone in the intracondillar region of femoral bone immediately after its creation is shown in FIG. 2D (x5).

FIG. 2E shows normal rat cranium. Defect area in parietal bone immediately after removal of 6×6 mm² full thickness bone segment is shown on Macro (FIG. 2F) and X-Ray (FIG. 2G) pictures. Microsection through the defect area is presented in FIG. 2H (x5).

Abbreviations: NC, normal cartilage; DA., defect area;

FIGS. 3A(1) to 3A(4) and B3(1) to B3(4): Influence of RTG polymers (N2 and N4) on correction of experimentally created calvarial defect by transplantation of demineralized bone matrix (DBM) and bone marrow cells (BMC).

FIGS. 3A(1), 3A(2), 3B(1) & 3B(2): X-Ray (FIGS. 3A(1), 3B(1)) and Macro (FIGS. 3A(2), 3B(2)) pictures of rats calvaria one month after transplantation of DBM-BMC complex supplemented with RTG polymeric materials N2 (FIGS. (FIGS. 3A(1), 3A(2)) and N4 ((FIGS. 3B(1), 3B(2)) into the area of experimentally created calvarial defect show complete regeneration of the bone.

FIGS. 3A(3), 3A(4), 3B(3) & 3B(4): Photomicrograph (x10) of cranial sections 30 days after the experimentally created calvarial defect followed by transplantation of DBM-BMC complex supplemented with RTG polymeric materials N2 (FIGS. 3A(3) and 3A(4)) and N4 (FIGS. 3B(3) and 3B(4)) show continuous layer of newly developed bone tissue with hematopoietic areas and active remodeling of the transplanted DBM particles. Cut edge of the bone can hardly be seen.

Abbreviations: D A., defect area; CE., cut edge;

FIGS. 4A, 4B, 4C(1) and 4C(2): Influence of RTG polymer N7 on correction of experimentally created calvarial defect by transplantation of demineralized bone matrix (DBM) and bone marrow cells (BMC).

FIGS. 4A & 4B: X-Ray (FIG. 4A) and Macro (FIG. 4B) pictures of rats calvaria one month after transplantation of DBM-BMC complex supplemented with RTG polymeric material N7 into the area of experimentally created calvarial defect show complete regeneration of the bone.

FIGS. 4C(1) & 4C(2): Photomicrograph (x10) of cranial sections 30 days after the experimentally created calvarial defect followed by transplantation of DBM-BMC complex supplemented with RTG polymeric material N7 show continuous layer of newly developed bone tissue with hematopoietic areas and active remodeling of the transplanted DBM particles.

Abbreviations: D A., defect area; CE., cut edge;

FIGS. 5A(1) to 5A(4) and 5B(1) to 5B(4): Influence of different RTG polymers on correction of experimentally created calvarial defect by transplantation of bone marrow cells (BMC).

FIGS. 5A(1), 5A(2), 5B(1) & 5B(2): X-Ray (FIGS. 5A(1), 5B(1)) and Macro (FIGS. 5A(2), 5B(2)) pictures of rats calvaria one month after transplantation of BMC supplemented only with RTG polymeric materials N2 (FIGS. 5A(1), 5A(2)) or N7 (FIGS. 5B(1), 5B(2)) into the area of experimentally created calvarial defect show absence of bone regeneration.

FIGS. 5A(3), 5A(4), 5B(3) & 5B(4): Photomicrograph (x10) of cranial sections 30 days after the experimentally created calvarial defect followed by transplantation of BMC supplemented only with RTG polymeric materials N2 (FIGS. 5A(3), 5A(4)) or N7 (FIGS. 5C(3), 5C(4)) confirms the absence of new bone development

Abbreviations: D A., defect area; CE., cut edge;

FIG. 6A-H: Photomicrographs of sagital knee joint sections one month after the experimentally created microfracture drilling defect followed by transplantation of demineralized bone matrix and bone marrow cells with RTG polymers N2 and N4.

FIGS. 6 A&B: Mixture of DBM particles with BMC supplemented with RTG polymer N2 was transplanted into defect area (x10 & x20). Active angiogenesis as well as partial degradation and remodeling of DBM particles are seen. No cartilage development can be observed, regenerating surface is built of connective tissue.

FIGS. 6 C&D: BMC supplemented with RTG polymer N2 were transplanted into defect area (x10 & x20). Regeneration of subchondral bone and hematopoietic cavities, no cartilage formation, regenerating surface is built of connective tissue.

FIGS. 6 E&F: Mixture of DBM particles with BMC supplemented with RTG polymer N4 was transplanted into defect area (x10 & x20). Partial degradation and remodeling of DBM particles are seen as well as development of hematopoietic cavities. No chondrogenesis can be seen; regenerating surface is built of connective tissue.

FIGS. 6 G&H: BMC supplemented with RTG polymer N4 were transplanted into defect area (x10 & x20). Regeneration of subchondral bone and hematopoiesis; no cartilage formation, regenerating surface is built of connective tissue.

FIG. 7A-D: Photomicrographs of sagital knee joint sections 4 weeks after the experimentally created microfracture drilling defect followed by transplantation of demineralized bone matrix and bone marrow cells with RTG polymer N7.

FIGS. 7A&B: Mixture of DBM particles with BMC supplemented with RTG polymer N7 was transplanted into defect area (x10 & x20). Extensively developing hyaline cartilage, as well as considerably degraded DBM particles can be seen. Regenerating surface is built of thick layer of hyaline cartilage.

FIGS. 7C&D: BMC supplemented with RTG polymer N7 were transplanted into defect area (x10 & x20). Complete regeneration of subchondral bone; surface of the damaged area comprises a mixture of connective tissue with cartilage cells.

FIG. 8A-H: Photomicrographs of sagital knee joint sections two months after the experimentally created microfracture drilling defect followed by transplantation of demineralized bone matrix and bone marrow cells with RTG polymers N2 and N4.

FIGS. 8 A&B: Mixture of DBM particles with BMC supplemented with RTG polymer N2 was transplanted into defect area (x10 & x20). Complete regeneration of subchondral bone is seen. No cartilage development can be observed, regenerating surface is built of connective tissue.

FIGS. 8 C&D: BMC supplemented with RTG polymer N2 were transplanted into defect area (x10 & x20). Regeneration of subchondral bone and hematopoietic cavities, no cartilage formation, regenerating surface is built of connective tissue.

FIGS. 8 E&F: Mixture of DBM particles with BMC supplemented with RTG polymer N4 was transplanted into defect area (xlO & x20). Almost degraded DBM particles and well developed subchondral bone and hematopoietic cavities are seen. No chondrogenesis, regenerating surface is built of connective tissue.

FIGS. 8 G&H: BMC supplemented with RTG polymer N4 were transplanted into defect area (x10 & x20). Regeneration of subchondral bone and hematopoiesis; no cartilage formation, regenerating surface is built of connective tissue.

FIG. 9A-D: Photomicrographs of sagital knee joint sections two months after the experimentally created microfracture drilling defect followed by transplantation of demineralized bone matrix and bone marrow cells with RTG polymer N7.

FIGS. 9A&B: Mixture of DBM particles with BMC supplemented with RTG polymer N7 was transplanted into defect area (x10 & x20). Continuous layer of young hyaline cartilage, as well as complete regeneration of subchondral bone can be seen, considerably degraded DBM particles are yet present.

FIGS. 9C&D: BMC supplemented with RTG polymer N7 were transplanted into defect area (x10 & x20). Complete regeneration of subchondral bone; surface of the damaged area comprises a mixture of connective tissue with cartilage cells.

FIG. 10A-D: Photomicrographs of sagital knee joint sections one month after the experimentally created microfracture drilling defect followed by transplantation of demineralized bone matrix and bone marrow cells without the addition of polymeric materials.

The Figure illustrates cases of incomplete repair of damaged osteochondral complex in the rat knee joint when the active composition comprising of DBM and BMC was applied alone without sufficient fixation with polymeric materials.

FIG. 10A: Most of BMC were washed out of the site of transplantation. Thus the transplanted area is packed with non-remodeled DBM particles, almost no new bone formation is observed.

FIG. 10B: Partial regeneration of subchondral bone, and surface hyaline cartilage, however some of DBM particles thrust into the joint surface preventing the formation of continuous cartilage layer.

FIG. 10C Total regeneration of subchondral bone and hematopoiesis, however the surface is occupied by DBM.

FIG. 10D: The active composition comprising of DBM and BMC was washed out of the damaged area, as a result, regenerating site is mostly filled by connective tissue.

### DETAILED DESCRIPTION OF THE INVENTION

The following abbreviations are utilized throughout this specification:
BM: bone marrow.
BMC: bone marrow cell(s).
BMP: bone morphogenetic protein.
DBM: demineralized bone matrix.
DTM: demineralized tooth matrix (DBM and DTM are used herein interchangingly).
LCM: Laser Capture Microdissection.
MBM: mineralized bone matrix.
PCR: polymerase chain reaction.
PIC: Picroindigocarmin, a dye used in histological staining.
RTG: Reverse thermogelating (polymer).

In search for improving the regeneration of damaged osteochondral complex in joint and cranio-facial-maxillary areas, by using a composition comprising BMC and DBM as a graft, the inventors have found that the addition of a highly viscous polymer to a composition comprising BMC and DBM results in the formation of a depot at the site of injection, preventing the migration of the BMC and DBM mixture away from the transplantation site. Moreover, the inventors have now interestingly proposed the use of not just a highly viscous polymer, which may not be syringeable at ambient temperatures, but to use a site-responsive polymer, for example a polymer with thermogelating properties, which is liquid and thus injectable at ambient temperature, yet gels at body temperature, forming the desired depot, which enables the maintenance of the integrity and stability of shape of the transplant, whilst providing mechanical properties essential to temporarily meet the requirements of the recipient throughout the period of tissue regeneration. The term body temperature as used herein is to be taken to mean a temperature of between 35° C. and 42° C., preferably about 37° C., particularly 37° C. The addition of the site-responsive, e.g. RTG polymer does not adversely affect the new tissue formation which follows a differentiation pathway producing different types of bone and cartilage, depending on the local conditions. Thus, the newly formed tissue meets precisely the local demands.

The present invention relates to compositions comprising a mixture of bone marrow cells (BMC) and demineralized bone matrix (DBM) and a site-responsive polymer, and to their novel uses in the transplantation of mesenchymal progenitor cells into joints and cranio-facial-maxillary bones.

Thus, in a first aspect, the present invention relates to a composition comprising bone marrow cells (BMC) and demineralized bone matrix (DBM) and a biocompatible, site-responsive polymer.

DBM is an essential ingredient in the composition of the invention in view of its advantageous ability to combine all the features needed for making it an excellent carrier for mesenchymal progenitor cells. The properties of DBM can be summarized as follows:

1. DBM can be a conductive scaffold essential for the engraftment, proliferation and differentiation of mesenchymal progenitor cells, in the course of bone and cartilage formation.

2. DBM is the natural source of BMPs, which are active in stimulating osteo- and chondrogenesis, thus also fulfilling the inductive function.

3. DBM is slowly biodegradable, the degradation time being compatible with the period of de novo chondro- and osteogenesis.

4. DBM has very low immunogenicity when used as a xenograft, and it is practically non-immunogenic when used in allogeneic combinations.

The site-responsive polymer may be a reverse thermogelating (RTG) polymer, or a polymer which responses to triggers other than or additional to body temperature, for example, pH, ionic strength, etc. In a particular embodiment, the site-responsive polymer may be a polymeric system comprising at least one silicon-containing reactive group. Polymeric components of this system may be RTG polymers, or otherwise responsive polymers, or combinations thereof.

The site-responsive polymers employed in the compositions of the invention are mainly: excellent compatibility with proliferation and differentiation of mesenchymal progenitor cells, in the course of bone and cartilage formation; absence of immunogenicity; liquid form at room temperature, allowing mixing it with the components of the active complex (DBM and BMC) and syringeability; and ability to develop high viscosity in response to a trigger at the administration site, e.g. body temperature, pH, ionic strength etc.

After its admixture with the active complex, the polymeric supplement is capable of rendering the complex sufficiently strong to maintain integrity and shape upon transplantation, as well as to provide biomechanical properties to the transplant, withstanding mechanical forces, during the period of new tissue formation.

The RTG polymer may be a random [-PEG6000-O—CO—(CH₂)₄—CO—O—PPG3000-]ₙpoly(ether-ester) or an alternating [-PEG6000-O—CO—O—PPG3000-]ₙ poly(ether-carbonate).

In a further embodiment, the invention can use a modified polymer, particularly an RTG or otherwise site-responsive silane-based polymer, displaying low viscosities at deployment time via minimally or non-invasive surgical procedures, and containing mono-, bi- or trifunctional silicone-containing reactive groups, most importantly alkoxysilane or silanol groups, capable of undergoing a condensation reaction at a predetermined body site, in the physiological conditions of humidity and temperature, whereby their molecular weight increases by virtue of their polymerization and/or crosslinking. Such polymers are described in detail in Israel Patent Application No. 155866, filed on May 12^{th} 2003, and fully incorporated herein by reference.

These polymers are components of an environmentally or site responsive polymeric system used as the RTG or otherwise responsive component comprised in the compositions of the invention. This system comprises a polymeric component containing reactive Si-based moieties capable of generating stable and inert Si—O—Si bonds in presence of water, primarily at a predetermined body site, resulting in said increase in the molecular weight of the polymeric system, producing a change in its rheological and mechanical properties. In some instances, these materials generate silicon-rich domains.

More specifically this polymeric system comprises one or more silicon-containing reactive groups capable of undergoing a condensation reaction primarily at a predetermined body site, in the presence of water and at body temperature, at an appropriate pH, wherein said reaction results in an increase in the molecular weight of the polymeric system due to polymerization and/or crosslinking and produces at least a partial change in the rheological and mechanical properties of said polymeric system.

In a preferred embodiment the polymeric system is biodegradable or selectively biodegradable, whereby the system clears from the administration site or reverts to an essentially un-polymerized or non-crosslinked state after a pre-determined time.

The polymeric system can also comprise additional reactive groups such as hydroxyl, carboxyl, thiol, amine, isocyanate, thioisocyanate or unsaturated moieties capable of polymerizing by a free radical polymerization, resulting in different interpenetrating networks (IPN's) and combinations thereof.

The polymeric system can comprise more than one component that forms covalent bonds between the different components or generates physical blends or interpenetrating or pseudo-interpenetrating networks and combinations thereof, at the predetermined body site.

In further preferred embodiments the composition of the invention may comprise, in addition to the responsive polymeric system or polymer and the BMC and DBM, at least one additional biomolecule to be delivered into the body such as elastin, collagenous material, albumin, a fibrinous material, growth factors, enzymes, hormones, living cells such as endothelial cells, hepatocytes, astrocytes, osteoblasts, chondrocytes, fibroblasts, miocytes, and combinations thereof.

In said more preferred embodiments the composition of the invention comprises also macro, micro or nano-sized solid component such as a polymer, a ceramic material, a metal, a carbon, a biological material, and combinations thereof, presenting the solid component different and various shapes such as particles, spheres, capsules, rods, slabs, fibers, meshes, ribbons, webs, non-woven structures, fabrics, amorphous lattice structures, filament wound structures, honeycomb or braided structures, and combinations thereof, wherein said solid component may be hollow, porous or solid, and combinations thereof.

In said more preferred embodiments the solid component possesses reactive moieties capable of reacting with the silicon-containing reactive groups present in said responsive polymeric system.

The responsive polymeric system comprised in the composition of the invention may generate a polymer selected from the group consisting of a linear polymer, a graft polymer, a comb polymer, a star-like polymer, a crosslinked polymer and combinations thereof.

In addition, the responsive polymeric system also comprises additional reactive groups selected from the group consisting of hydroxyl, carboxyl, thiol, amine, isocyanate, thioisocyanate and double bond-containing active groups and combinations thereof.

In even more preferred embodiments the responsive polymeric system is a low molecular weight polymer capable of being deployed at a predetermined body site by minimally invasive procedures, such as polyoxyalkylene, polyester, polyurethane, polyamide, polycarbonate, polyanhydride, polyorthoesters, polyurea, polypeptide, polyalkylene, polysaccharide and combinations thereof.

In even more preferred embodiments the responsive polymeric system is also capable of undergoing a transition that results in a sharp increase in viscosity in response to a predetermined trigger such as temperature, pH, ionic strength, at a predetermined body site, resulting in an increase in the viscosity of said responsive polymeric system by at least about two times, wherein said transition takes place before and/or during and/or after the chemical triggering reaction.

In even more preferred embodiments the responsive polymeric system comprises water or a water-based solvent such as ethanol or isopropyl alcohol.

In especially preferred embodiments the responsive polymeric system is a polyoxyalkylenc polymer, a block copolymer comprising polyethylene oxide (PEO) and polypropylene oxide (PPO) selected from a group consisting of a diblock, a triblock or a multiblock, a segmented block copolymer comprising polyethylene oxide (PEO) and polypropylene oxide (PPO) chains, wherein said PEO and PPO chains are connected via a chain extender, a poly(alkyl-co-oxyalkylene) copolymer having the formula R—(OCH₂CH)ₙ—OH, where R is an hydrophobic monofunctional segment selected from a group consisting of poly(tetramethylene glycol), poly(caprolactone), poly(lactic acid), poly(siloxane) and combinations thereof, a poly(alkyl-co-oxyalkylene) copolymer having the formula [—R′—(OCH₂CH)ₙ—O]ₚH, where R′ is a bifunctional or multifunctional hydrophobic segment, a poly(N-alkyl substituted acrylamide), preferably poly(N-isopropyl acrylamide), cellulose and cellulose derivatives, alginates and its derivatives, hyaluronic acid and its derivatives, collagen, gelatin, chitosan and its derivatives, agarose, water soluble synthetic, semi-synthetic or natural oligomers and polymers selected from a groups consisting of oligoHEMA, polyacrylic acid, polyvinyl alcohol, glycerol, polyethylene oxide, TMPO, oligo and polysaccharides, oligopeptides, peptides, proteins, enzymes, growth factors, hormones, drugs and combinations thereof.

Preferably said chain extender is phosgene, aliphatic or aromatic dicarboxylic acids or their reactive derivatives such as acyl chlorides and anhydrides or other molecules able to react with the OH terminal groups of the PEO and PPO chains, such as dicyclohexylcarbodiimide (DCC), aliphatic or aromatic diisocyanates such as hexamethylene diisocyanate (HDI) or methylene bisphenyldiisocyanate (MDI) or cyanuric chloride or any other bifunctional or multifunctional segment, and/or combinations thereof.

In even more preferred embodiments the responsive polymeric system contains other polymers that are responsive to other stimuli selected from a group consisting of temperature, pH, ionic strength, electric and magnetic fields, ultrasonic radiation, fluids and biological species and combinations thereof.

The biologically and/or pharmacologically active components contained in the compositions of the invention, particularly DBM, DTM and BMC, can be delivered into the body following a unimodal or multimodal time-dependent release kinetics, as the molecular weight of the polymeric system as well as its rheological and mechanical properties change at the predetermined body site.

The biologically or pharmacologically active molecule/s to be delivered into the body, may be covalently bound to the silicon groups, affording homogeneously distributed reservoirs.

In even more preferred embodiments the responsive component can be used as sealant, as coating and lubricant, as transient barrier for the prevention of post-surgical adhesions, as matrix for the unimodal or multimodal controlled release of biologically active agents for the desired tissue engineering.

In further preferred embodiments the silicon moieties serve as nuclei for the deposition or crystallization of various materials preferably hydroxyapatite or other calcium phosphate derivatives for bone regeneration induction at a predetermined body site.

The novel, tailor-made polymeric systems used in the compositions of the present invention display advantageous properties unattainable by the prior art by capitalizing, in a unique and advantageous way, on the low viscosity of the polymeric system at administration, and the molecular weight increase and/or crosslinking in situ, with or without additional additives or initiator/catalyst systems.

Compositions according to this invention are suitable to be used in the human body, preferably in applications where the combination of ease of administration and enhanced initial flowability and thus syringeability, on one hand, and post-implantation high viscosity and superior mechanical properties, on the other hand, are required.

The responsive polymeric systems to be contained in the compositions of the inventions have important advantages in a variety of important biomedical applications, such as in non-invasive surgical procedures, in the prevention of post-surgical adhesions and in the Tissue Engineering field, designed to cover a broad range of mechanical properties. In the case of biodegradable systems, these materials are engineered to display different degradation kinetics.

The polymeric system of the invention can contain hydrolytically unstable segments along the polymeric backbone, thus allowing for fine tuning of both the degradation rate of the polymer molecule as well as controlling the stability of the whole system and its rheological properties. These compositions can be conferred with specific biological functions by incorporating biomolecules of various types, physically (by blending them into the polymeric system) or chemically (by covalently binding them to the polymer). It is an additional object of the invention to incorporate cells of various types into these materials, for them to perform as RTG-displaying matrices for cell growth and tissue scaffolding.

In a second aspect, said composition comprising BMC and DBM and the site-responsive polymer is for use in transplantation of mesenchymal cells and/or mesenchymal progenitor cells into a joint and/or a cranio-facial-maxillary area of a subject in need, wherein said subject is a mammal, preferably a human.

It is an object of the present invention to provide the said composition for transplantation of BMC into damaged joints and/or a cranio-facial-maxillary area for the replacement and/or restoration of hyaline cartilage and bone, originating from the mesenchymal precursor cells existing in the transplanted BMC.

The DBM comprised within the composition of the invention is preferably of vertebrate origin, and may be of human origin.

The DBM comprised within the composition of the invention is preferably in powder form. The particle size of the DBM may be about 50 to 2500μ.

Preferably, said particle size is about 250 to 500μ. The most preferable particle size will depend on the specific needs of each case.

In another embodiment, the composition of the invention is for restoring and/or enhancing the formation of a new hyaline cartilage and bone structure.

As described in WO02/070023, the idea underlying the administration of a mixture of DBM with BMC is that BMC may provide a source for mesenchymal stem cells, which are capable of inducing osteo- and chondrogenesis. Thus, as described in said application, when a BMC suspension in admixture with DBM powder was administered directly into either a joint bearing a damage in the osteo-chondral complex, or in the cranium of an animal with a partial bone defect in the parietal bone, significant restoration occurred.

The idea underlying the present invention is that supplementing the active composition of BMC and DBM with polymeric materials exhibiting high viscosity at body temperature, and/or in response to other environmental trigger within the patient's body such as pH or ionic strength, results in improving the ability to maintain the integrity and shape of the transplanted complex, whilst providing mechanical properties essential to temporarily meat the requirements of the organism (such as withstanding physical pressures etc.) throughout the period of tissue regeneration.

Thus, as described in the following examples, when a mixture of BMC suspension and DBM powder, in admixture with site-responsive polymeric materials was administered directly into either a joint damaged in the osteo-chondral complex, or in the cranium of an animal with a critical size bone defect, significant restoration occurred. Treated recipients were mobile with no need for fixation of the joints, and full restoration of the anatomic structure of the treated joint was accomplished. This is a major advantage of the compositions of the present invention, since it obviates the need for long periods of rest following transplantation/implantation. Likewise, newly reconstituted parietal bone replacing surgically removed parietal bone in the skull showed normal remodeling. In the damaged joint, there was formation of subchondral bone structure and hyaline cartilage, and in the cranial defect, new flat bone was formed.

The present compositions obviate the need for using biological say fixation and/or strengthening, necessary in the application of the earlier BMC-DBM complexes. In addition, the present compositions provide a scaffold and template for molding any desirable shape and structure, according to the location of the implant. Such a scaffold provides an immediate mechanical support that minimizes the need for immobilization of the recipient following therapy. In addition the feasibility of injection of the mixture into the joint, may avoid the need for open surgery, thus minimizing iatrogenic damage, discomfort, need for immobilization, scar formation and risk of infections.

In a further embodiment, the composition of the invention is intended for the treatment of a patient suffering from any one of a hereditary or acquired bone disorder, a hereditary or acquired cartilage disorder, a malignant bone or cartilage disorder, metabolic bone diseases, bone infections, conditions involving bone or cartilage deformities and Paget's disease. Said disorders are listed in detail in Table 1. Additionally, the invention is also intended for the treatment of a patient in need of any one of correction of complex fractures, bone replacement, treatment of damaged or degenerative arthropathy and formation of new bone in plastic or sexual surgery.

TABLE 1

| Congenital and | | | |
|---|---|---|---|
| Hereditary Bone | | Metabolic Bone | Non-neoplastic Disorders |
| Disorders | Bone Infections | Diseases | of the Bone |
| Achondroplasia | Hematogenous | Osteoporosis | Fibrous Dysplasia of the |
| | (Pyogenic) | | Bone |
| | Osteomyelitis | | |
| Osteogenesis | Osteomyelitis from | Rickets and | Fibrous Cortical Defect |
| Imperfecta (Brittle | a Contiguous | Osteomalacia | and Non-ossifying |
| Bones, Fragilitas | Infection | | Fibroma |
| Ossium) | | | |
| Osteopetrosis | Osteomyelitis from | Bone Changes in | Solitary Bone Cyst |
| (Marble Bone | an Introduced | Hyperparathyroidism | (Unicameral Bone Cyst) |
| Disease, | Infection | (Generalized | |
| Osteosclerosis) | | Osteitis, Cystic | |
| | | Fibrosis, Von | |
| | | Recklinghausen's | |
| | | Bone Disease) | |
| Hereditary | Bone Tuberculosis | Renal | Aneurysmal Bone Cyst |
| Multiple Exotosis | | Osteodystrophy | |
| (Osteochondromatosis) | | | |
| Enchondromatosis | Bone Syphilis | Paget's Disease of | Eosinophilic Granuloma of |
| (Ollier's Disease) | | Bone (Osteitis | Bone |
| | | Deformans) | |
| | Bone Fungus | | Bone Lesions of Gaucher's |
| | Infections | | Disease |

In a yet further embodiment, the composition of the invention may further optionally comprise a pharmaceutically acceptable carrier or diluent, as well as additional active agents, as described above.

Pharmaceutically acceptable (or physiologically acceptable) additive, carrier and/or diluent mean any additive, carrier or diluent that is non-therapeutic and non-toxic to recipients at the dosages and concentrations employed, and that does not affect the pharmacological or physiological activity of the active agent.

The preparation of pharmaceutical compositions is well known in the art and has been described in many articles and textbooks, see e.g., Remington's Pharmaceutical Sciences, Gennaro A. R. ed., Mack Publishing Company, Easton, Pa., 1990, and especially pages 1521-1712 therein.

Active agents of particular interest are those agents that promote tissue growth or infiltration, such as growth factors. One example is BMPs, which may enhance the activity of the composition of the invention. Other exemplary growth factors for this purpose include epidermal growth factor (EGF), osteogenic growth peptide (OGP), fibroblast growth factor (FGF), platelet-derived growth factor (PDGF), transforming growth factors (TGFs), parathyroid hormone (PTH), leukemia inhibitory factor (LIF), insulin-like growth factors (IGFs), and growth hormone. Other agents that can promote bone growth, such as the above-mentioned BMPs, osteogenin [Sampath et al. (1987) Proc. Natl. Acad. Sci. USA 84:7109-13] and NaF [Tencer et al. (1989) J. Biomed. Mat. Res. 23: 571-89] are also preferred.

Other active agents may be anti-rejection or tolerance inducing agents, as for example immunosuppressive or immunomodulatory drugs, which can be important for the success of bone marrow allografts or xenografts transplantation.

Alternatively, said active agents may be for example antibiotics, provided to treat and/or prevent infections at the site of the graft. On the same token, anti-inflammatory drugs can also be added to the composition of the invention, to treat and/or prevent inflammations at the site of the graft. Said inflammations could be the result of for example rheumatoid arthritis, or other conditions.

In addition to the site-responsive polymers, the compositions of the invention may contain other polymeric or biodegradable materials, which are pharmaceutically acceptable carriers and diluents. Biodegradable films or matrices, semi-solid gels or scaffolds include calcium sulfate, tricalcium phosphate, hydroxyapatite, polylactic acid, polyanhydrides, bone or dermal collagen, fibrin clots and other biologic glues, pure proteins, extracellular matrix components and combinations thereof. Such biodegradable materials may be used in combination with non-biodegradable materials, to provide additional desired mechanical, cosmetic or tissue or matrix interface properties.

In preferred embodiments, the composition of the invention contains BMC-DBM mixture and polymeric material at a ratio of from 5:1 to 1:5, preferably between 3:1 and 1:2, most preferably at a ratio of 2 part BMC-DBM mixture to 1 part of polymeric material in fluid form (volume:volume). The absolute number of BMC, as well as the volumes of DBM and polymeric material are dependent on the size of the joint to be rehabilitated or the size (surface, shape and thickness) of the bone to be replaced, while the cell concentrations of BMC suspensions ranging from 1×10⁶/ml to 1×10⁸/ml and DBM at a ratio of from 1:1 to 20:1, preferably between 2:1 to 9:1, most preferably the composition of the invention is at a ratio of 4 parts BMC concentrate to 1 part of DBM in powder form (volume:volume).

In order to obtain the desired viscosity following injection, the concentration of the polymer in the compositions of the invention is to be carefully adjusted. The optimal concentration will be achieved using Viscosity vs. Concentration calibration curves. The results presented herein show that the concentration of the polymer is to be optimized, and generally, very high concentration are to be avoided, because they may prevent the desired flow of biological nutrients and molecules and thus adversely affect the induction process.

In another aspect, the present invention relates to a method for transplantation of a mixture comprising BMC with DBM and a site-responsive polymer, optionally further comprising pharmaceutically acceptable carrier or diluent, into a joint and/or a cranio-facial-maxillary bone area of a subject in need, wherein said method comprises introducing into said joint or bone the composition of the invention.

The composition of the invention, which possesses all the essential features for accomplishing local bone formation wherever it is implanted, can be efficiently applied for all kinds of bone repair or substitution, especially in places lacking or deprived of mesenchymal stem cells. Amongst the most problematic places in this sense are joints, cranio-facial-maxillary areas and different kinds of segmental bony defects. Thus, the present invention may be explained as a complex graft, comprising all necessary components, and which its implantation into a damaged area is sufficient for regeneration or substitution of removed, damaged or destroyed cartilage and/or bone.

In a first embodiment of the method of the invention, the mixture is administered by any one of the following procedures, injection, minimally invasive arthroscopic procedure, or by surgical arthroplasty into the site of implantation, wherein said method is for support or correction of congenital or acquired abnormalities of the joints, cranio-facial-maxillary bones, orthodontic procedures, bone or articular bone replacement following surgery, trauma or other congenital or acquired abnormalities, and for supporting other musculoskeletal implants, particularly artificial and synthetic implants.

Thus, in a further aspect, the invention relates to a method of treating a damaged or degenerative arthropathy associated with malformation and/or dysfunction of cartilage and/or subchondral bone in a mammal in need of such treatment, comprising administering into an affected joint or bone of said mammal a mixture comprising BMG with DBM, together with a site-responsive polymer, said mixture optionally further comprising a pharmaceutically acceptable carrier or diluent and/or additional active agents.

As demonstrated in the following examples, the addition of the RTG polymer did not adversely affect the process of induced development (i.e. proliferation and differentiation) of mesenchymal progenitor cells present within the BMC/DBM(DTM)/RTG mixture can accomplish bone and cartilage formation wherever the mixture is transferred to. The findings presented herein indicate that administration of the composition of the invention into a damaged area of the joint, results in generation of new osteochondral complex consisting of articular cartilage and subchondral bone, same as in the absence of the RTG. When administered into an experimentally created calvarial defect, the composition of the invention results in generation of full intramembranous bone development at the site of transplantation. New tissue formation follows a differentiation pathway, producing different types of bone and cartilage, depending on the local conditions. Thus, the newly formed tissue meets precisely the local demands.

The procedure of applying the composition of the invention into a damaged joint or cranial area comprises the following steps:

1. Selecting the source for BMC. The donor may be allogeneic or the BMC may be obtained from the same treated subject (autologous transplantation).

2. Selecting the source of DMB. The DBM may be supplied commercially and since it is not immunogenic, there are no limitations for a specific donor. DMB may be in powder, granules or in slice form. The particle size of the DBM may be about 50 to 2500μ. Preferably, said particle size is about 250 to 500μ. The most preferable particle size will depend on the specific needs of each case.

3. Preparing a composition comprising a suspension of BMC, at a cell concentration ranging from 1×10⁶/ml to 4×10¹⁰/ml and mixing it with DBM at a ratio of from 1:1 to 20:1, preferably between 2:1 to 9:1, most preferably the composition of the invention is at a ratio of 4 parts BMC concentrate to 1 part of DBM in powder form (volume:volume). MBM may be used instead of DBM. If so desired, BMP may optionally be included in the composition.

4. Adding to the composition obtained in step 3 a site-responsive polymer, at an optimal concentration for the site-responsive polymer used.

5. Administering the composition obtained in step 4 into a subject in need either through a syringe (non-invasive injection), closed arthroscopy or open surgical procedure. Alternatively, the composition may be administered so that it is encapsulated within normal tissue membranes. Still alternatively, the composition may be contained within a membranous device made of a selective biocompatible membrane that allows cells, nutrients, cytokines and the like to penetrate the device, and at the same time retains the DBM articles within the device. Such a membranous device, bone strips or additional scaffolds are preferably surgically introduced.

In a yet further aspect, the present invention relates to a non-invasive transplantation method comprising introducing a graft into a joint or a cranio-facial-maxillary bone of a subject in need, wherein said graft comprises a mixture of BMC and DBM together with a site-responsive polymer.

In the examples presented herein (see Examples), the inventors show that administration of the composition of the present invention (e.g. BMC in admixture with DBM and a site-responsive polymer, as in Example 3) into a damaged area of the joint is essential and sufficient for the generation of new osteochondral complex, consisting of articular cartilage and subchondral bone, at the site of transplantation. The newly formed donor-derived osteochondral complex was capable of long-term maintenance, remodeling and self-renewal, as well as carrying out specific functions of joint surface, such as motion and weight bearing.

In an even further aspect, the present invention relates to the use of a composition comprising BMC and DBM together with a site-responsive polymer as a graft of mesenchymal and/or mesenchymal progenitor cells for transplantation into a mammal, wherein said mammal is preferably a human. The transplantation is to be performed into a joint or into a cranio-facial-maxillary bone, for the development of new bone and/or cartilage. The graft of said transplantation may also be for supporting orthodontic procedures for bone augmentation caused by aging, or by congenital, acquired or degenerative processes.

Furthermore, the composition used in said transplantation is intended for the treatment of a patient suffering from any one of a hereditary or acquired bone disorder, a hereditary or acquired cartilage disorder, a malignant bone or cartilage disorder, conditions involving bone or cartilage deformities and Paget's disease. In addition, said composition is intended for the treatment of a patient in need of any one of correction of complex fractures, bone replacement, treatment of damaged or degenerative arthropathy and formation of new bone in plastic or sexual surgery.

The method of the invention may also be used to induce or improve the efficiency of bone regeneration in damaged cranio-facial-maxillary areas, for therapeutic and cosmetic purposes.

In one embodiment, the composition used in the invention further comprises an additional active agent.

In another embodiment, the DBM comprised within the composition used in the invention are of vertebrate origin, and they may be of human origin. Moreover, said DBM is preferably in powder form.

In an additional aspect, the present invention concerns the use of a mixture of BMC with DBM together with a site-responsive polymer in the preparation of a graft for the treatment of a bone or cartilage disorder, and/or for support of musculoskeletal implants, as a scaffold to enforce metal implants, joints, etc. that may become loose with time, or to provide a continuously adapting “biological scaffold” to support such non-biological implants. Alternatively, the invention could be for the support of limb transplants, especially in the articular/bone junction.

Lastly, the present invention provides a kit for performing transplantation into a joint or for reconstruction of cranio-facial-maxillary bone area, long bones, pelvis, spines or for dental support through alveolar bone of maxilla and mandibula augmentation or for creation of an artificial hematopoietic bone of a mammal of BMC in admixture with DBM and a site-responsive polymer, wherein said kit comprises:
(a) DBM in powder or a compacted form (e.g. strings for reconstruction of tendons, or larger particles of DBM for reconstruction of large bone area);
(b) a site-responsive polymer;
(c) a BM aspiration needle;
(d) an intra-osseous bone drilling burr;
(e) a needle with a thick lumen for infusion of viscous bone marrow-DBM—site-responsive polymer mixture;
(f) a 2-way lumen connector for simultaneous mixing of BMC with DBM and site-responsive polymer and diluent;
(g) a medium for maintaining BMC; and optionally
(h) additional factors stimulating osteogenesis
(i) cryogenic means for handling and maintaining BMC or BMC together with DBM1.

The kit of the invention may optionally further comprise a carrier and/or a diluent for the BMC and DBM mixture, and for the site-responsive polymer.

The present inventors have previously concluded [PCT/IL02/00172] that transplantation of multipotent mesenchymal stem cells, and not of differentiated bone or chondrocytes, for remodeling and restoration of a healthy joint or cranio-facial-maxillary structure in arthropathy, is especially important for the following reasons:

(1) Chondrocytes, as well as the cells transferred within a bone transplant are already fully differentiated cells, with relatively low metabolic activity and limited self-renewal capacity that may be sufficient to maintain healthy cartilage or bone, but is certainly insufficient for the development of large areas of bone or of hyaline cartilage de novo.

(2) Most frequently in joints, both cartilage and subchondral bone are damaged. Thus, even a successfully developed new hyaline cartilage is unlikely to be maintained for long if the subchondral bone is left damaged. Based on these findings, it was observed in the following examples that mesenchymal stem cells present in bone marrow, if transplanted under the appropriate conditions, will create a self-supporting osteochondral complex providing healthy joint surface.

It is not yet clear what makes multipotential mesenchymal stem cells, under the influence of DBM, to choose between an osteogenic and a chondrogenic differentiation pathway. It has however been reported that the ratio of cartilage to bone production depends in particular on the site of DBM implantation, which is naturally influenced by the local conditions [Inoue, T. et al. (1986) J Dent Res 65(1):12-22], such as the local source of mesenchymal cells and blood supply [Reddi, A. H. and Huggins, C. H. (1973) P.S.E.B.M. 143:634-637]. Low oxygen tension favors chondrogenesis [Bassett, C. A. L. (1962) J Bone Joint Surg 44A:1217], most likely due to the low O₂ tension in poorly vascularized cartilage [Sledge, C. B. and Dingle, J. T. (1965) Nature (London) 205: 140]. Interestingly, a successful substitution of anterior cruciate ligament (ACL) by demineralized cortical bone matrix has been reported in a goat model [Jackson, D. W. et al. (1996) Amer J Sports Medicine 24(4):405-414]. The remodeling process included new bone formation within the matrix in the osseous tunnels and a ligament-like transition zone developing at the extra-articular tunnel interface [Jackson, D. W. et al. (1996) id ibid.]. Taking into consideration that hyaline cartilage is naturally developed and maintained only in the joints, where contact with synovial membranes and lubrication with synovial fluid is available and probably essential, it seems reasonable to assume that the environmental conditions in the joint play a major role in enhancing chondrogenesis.

In the following examples the inventors have shown, that a graft composed of DBM and bone marrow cells, together with a site-responsive polymer, transplanted into a damaged joint or cranial bone, led to successful replacement of damaged cartilage and subchondral bone. This was the result of osteogenesis on the side of contact with bone and chondrogenesis on the free joint surface, thus the physiological environmental conditions favored osteogenesis or chondrogenesis, respectively. The same kind of a graft composed of DBM and bone marrow cells together with a site-responsive polymer transplanted into experimentally created partial bone defect in the parietal bone of the cranium led to successful replacement of the removed part of the bone. Thus, the new tissue formation follows a differentiation pathway, producing different types of bone and cartilage depending on the local conditions, such that the newly formed tissue meets precisely the local demands.

The addition of a site-responsive polymer to the BMC/DBM preparation therefore results in a composition that is injectable a room temperature, but is highly viscous at body temperature and thus forms a depot upon injection, can be employed in non- or minimally invasive techniques and prevent migration of the bioactive components away from the injection site.

Many publications are referred to throughout this application. The contents of all of these references are fully incorporated herein by reference.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

1. Animals

8 weeks old C57BL/6 male mice and Lewis male rats with body weight of 180-200 g were used as the donors of bones (for matrix preparation) and BMC. Animals from the same batches were used as graft recipients.

2. Preparation of Demineralized Bone Matrix (DBM)

Demineralized bone matrix (DBM) was prepared as described [Reddi and Huggins (1973) id ibid.] with the inventors' modification. Diaphyseal cortical bone cylinders from Lewis rats were cleaned from bone marrow and surrounding soft tissues, crumbled and placed in a jar with magnetic stirring. Bone chips were rinsed in distilled water for 2-3 hrs; placed in absolute ethanol for 1 hr and in diethyl ether for 0.5 hr, then dried in a laminar flow, pulverized in a mortar with liquid nitrogen and sieved to select particles between 400 and 1,000μ. The obtained powder was demineralized in 0.6M HCl overnight, washed for several times to remove the acid, dehydrated in absolute ethanol and diethyl ether and dried.

With the exception of the drying step, all steps of the procedure were performed at 4° C., to prevent degradation of Bone Morphogenetic Proteins (BMP) by endogenous proteolytic enzymes. The matrices were stored at −20° C.

3. Preparation of the Implanted Material

Preparation of Donor BMC Suspensions for Transplantation:

The femurs of donor mice or rats were freed of muscle. Marrow plugs were mechanically pressed out of the femoral canal by a mandrin. Highly concentrated single cell suspensions of BMC were prepared by dissolving 4-5 femoral plugs into 100 μl of RPMI 1640 medium (Biological Industries, Beit Haemek, Israel), and passing the cells through the needle several times to dissolve the bone marrow tissue into a single-cell suspension. The number of nucleated cells per femoral bone marrow plug is rather stable (about 10⁷ cells/plug for a C57B/6 male, 8 week old mouse). Several reproducible verifications have shown that BMC prepared for transplantation in a form of a single cell suspension contains an approximate concentration of 3×10⁸ cells/ml.

4. Preparation of Polymeric Materials.

Polymer N2

The material is the commercially available Pluronic F-127 Sigma (Catalogue No. P-2443).

Polymer N4

Random [-PEG6000-O—CO—(CH₂)₄—CO—O—PPG3000-]ₙ poly(ether-ester)

15.3 grams (0.003 mol) of dry PEG6000 (molecular weight 6,000) and 7.4 g (0.003 mol) of PPG3000 were dissolved in 30 ml dry chloroform in a 250 ml flask. 3.2 g pyridine were added to the reaction mixture. Then 2.2 g adipoyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

Polymer N7

Alternating [-PEG6000-O—CO—PPG3000-]ₙ poly(ether-carbonate)

i) Synthesis of Phosgene and Preparation of the Chloroformic Solution

The phosgene was generated by reacting 1,3,5 trioxane (15 g) with carbon tetrachloride (100 g) using aluminum trichloride (30 g) as the catalyst. The phosgene vapors were bubbled in weighed chloroform and the phosgene concentration (w/w) was calculated by weight difference (between 9% and 11%). Due to phosgene's high toxicity, the solution was handled with extreme care and all the work was conducted under a suitable hood.

ii) Synthesis of PEG6000 dichloroformate (ClCO—O-PEG6000-O—COCl)30.3 grams of dried PEG6000 (molecular weight 6,000) were dissolved in 50 ml dried chloroform in a 250 ml flask. 66 gram of chloroformic solution of phosgene 3% w/w (100% molar excess to PEG) were added to the PEG and the mixture was allowed to react at 60° C. for 4 h with magnetic stirring and a condenser in order to avoid solvent and phosgene evaporation. The reaction flask was connected to a NaOH trap (20% w/w solution in water/ethanol 1:1) in order to trap the phosgene that could be released during the reaction. Once the reaction was completed, the system was allowed to cool down to RT and the excess of phosgene was eliminated by vacuum. The FT-IR analysis showed the characteristic peak at 1777 cm⁻¹ belonging to the chloroformate group vibration.

iii) Synthesis of alternating [-PEG6000-O—CO—O—PPG3000-]ₙ poly(ether-carbonate)

15.2 grams of dried PPG3000 (molecular weight 3,000) were added to ClCO-PEG6000-COCl produced in a) at RT. The mixture was cooled to 5° C. in an ice bath and 6.3 grams pyridine dissolved in 20 ml chloroform were added dropwise over a 15 min period. Then, the temperature was allowed to heat up to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained. The material displayed a melting endotherm at 53.5° C. and the FT-IR analysis showed the characteristic carbonate group peak at 1746 cm⁻¹. The molecular weight of the polymer produced was Mₙ 36,400 (M_{w}/Mₙ=1.28), as determined by GPC.

### Synthesis of Pluronic F127 di-(3-isocyanatopropyl)triethoxysilane (F127 di-IPTS)

25.2 g (0.002 mol) Pluronic F127 (molecular weight 12,600) were poured in a three-necked flask and dried at 120° C. under vacuum for 2 hours. Then, 1.2 g (0.005 mol) IPTS and 0.1 g (3.10⁻⁴ mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in a petroleum ether 40-60 (400 ml). Finally, the F127 derivative was washed repeatedly with portions of petroleum ether and dried in vacuum at RT. The synthesis is presented in Scheme 1.

Three compositions comprising F127 di-IPTS at different concentrations were used in the following Examples, designated #21, #22 and #23 (see FIG. 11). When subjected to body temperature (37° C.), their polymerization process includes two stages. The first comprises the ethoxysilane group hydrolysis to silanol groups and the second the condensation of the generated silanol groups to form Si—O—Si bonds.

### Synthesis of Pluronic F38 di-(3-isocyanatopropyl)triethoxysilane (F38 di-IPTS)

20.1 g (0.004 mol) Pluronic F38 (molecular weight 4,600) were poured in a three-necked flask and dried at 120° C. under vacuum for 2 hours. Then, 2.6 g (0.01 mol) IPTS and 0.2 g (3.10⁻⁴ mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in a petroleum ether 40-60 (400 ml). Finally, the F38 derivative was washed repeatedly with portions of petroleum ether and dried in vacuum at RT.

Poly(ethylene glycol) ME=400 di-(3-isocyanatopropyl)triethoxysilane (PEG400 di-IPTS)

5.1 g (0.013 mol) PEG400 were poured in a three-necked flask and dried at 120° C. under vacuum for 1 hours. Then, 7.6 g (0.019 mol) IPTS and 1.5 g (0.004 mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in a petroleum ether 40-60 (400 ml). Finally, the PEG400 di-IPTS was washed repeatedly with portions of petroleum ether and dried in vacuum at RT. Whereas the material was a liquid at 37° C., after incubation at this temperature a brittle and transparent film was formed.

Poly(ethylene glycol) MW=600 di-(3-isocyanatopropyl)triethoxysilane (PEG600 di-IPTS)

20.1 g (0.034 mol) PEG600 were poured in a three-necked flask and dried at 120° C. under vacuum for 1 hours. Then, 18.3 g (0.007 mol) IPTS and 1.5 g (0.004 mol) SnOct₂ were added to the reaction mixture and reacted at 80° C. for one hour, under mechanical stirring (160 rpm) and a dry nitrogen atmosphere. The polymer produced was dissolved in chloroform (30 ml) and precipitated in a petroleum ether 40-60 (400 ml). Finally, the PEG600 di-IPTS was washed repeatedly with portions of petroleum ether and dried in vacuum at RT. Whereas the material was a liquid at 37° C., after incubation at this temperature a brittle and transparent film was formed.

Poly(ethylene glycol) MW=1000 di-(3-isocyanatopropyl)triethoxysilane (PEG1000 di-IPTS)

10.2 g (0.010 mol) PEG1000 were poured in a three-necked flask and dried at 120° C. under vacuum for 1 hours. Then, 5.4 g (0.022 mol) IPTS and 0.5 g (0.001 mol) SnOct₂ were added to the reaction mixture and reacted at 80° C. for one hour, under mechanical stirring (160 rpm) and a dry nitrogen atmosphere.

The polymer produced was dissolved in chloroform (30 ml) and precipitated in a petroleum ether 40-60 (400 ml). Finally, the PEG1000 di-IPTS was washed repeatedly with portions of petroleum ether and dried in vacuum at RT. Whereas the material was a paste at 37° C., after incubation at this temperature a brittle and transparent film was formed.

Polycaprolactone MW=530 di-(3-isocyanatooropyl)triethoxysilane (PCL530 di-IPTS)

a) Synthesis of PCL530 di-IPTS

20.2 g of PCL530 were dried for 1 h at 120° C. in vacuum. Then the temperature was stabilized at 80° C. and 1.9 g catalyst and 22.4 g (0.09 mol) IPTS were added. The reaction continued for 1 h at this temperature in N₂ atmosphere. Finally, the reaction mixture was cooled to RT, washed with 50 ml of petroleum ether 40-60 and dried at RT in vacuum for 24 hours. The material was a slightly yellow liquid at RT.

b) Crosslinking of PCL530 di-IPTS

5 g of PCL530 di-IPTS synthesized in a) were poured in a 25 ml vial (30 mm diameter) and heated at 37° C. Then 1 ml PBS (pH 7.4 0.1 M) were added onto the material. The system was incubated at 37° C. The resulting material was yellow and transparent.

Polycaprolactone MW=2000 di-(3-isocyanatopropyl)triethoxysilane (PCL2000 di-IPTS)

a) Synthesis of PCL2000 di-IPTS

10.2 g of PCL2000 (0.005 mol) were poured in 100 ml flask and heated to 80° C. and 0.25 g catalyst and 3.1 g (0.09 mol) IPTS were added. The reaction continued for 1 h at this temperature in dry N₂ atmosphere. Finally, the reaction mixture was cooled to RT, washed with 50 ml of petroleum ether 40-60 and dried at RT in vacuum for 24 hours. The material is a white wax at. RT.

b) Crosslinking of PCL2000 di-IPTS

5 g of PCL2000 di-IPTS synthesized in a) were heated to 70° C. and poured in a 25 ml vial (30 mm diameter) and heated at 37° C. Then 1 ml PBS (pH 7.4 0.1 M) were added onto the material. The system was incubated at 37° C. The resulting material was a white and hard product.

### Trimethylolpropane ethoxylate MW=10 14 tri-(3-isocyanatopropyl)triethoxysilane (TMPE1014 tri-IPTS)

a) Synthesis of TMPE1014 tri-IPTS

5.1 g of (0.005 mol) TMPE1014 were dried for 1 h at 120° C. in vacuum. Then the temperature was stabilized at 80° C. and 0.4 g catalyst and 4.4 g (0.02 mol) IPTS were added. The reaction continued for 1 h at this temperature. Finally, the reaction mixture was cooled to RT, washed with 50 ml of petroleum ether 40-60 and dried at RT in vacuum for 24 hours. The material was a liquid at RT.

b) Crosslinking of TMPE1014 tri-IPTS

5 g of TMPE1014 tri-IPTS synthesized in a) were poured in a 25 ml vial (30 mm diameter) and heated at 37° C. Then 1 ml PBS (pH 7.4 0.1 M) were added onto the material. The system was incubated at 37° C. The resulting material was a transparent product.

PCL530 di-IPTS/PCL2000 di-IPTS crosslinked copolymer

The synthesis of PCL530 di-IPTS and PCL530 di-IPTS was described above. 5 g of material with different PCL530 di-IPTS/PCL2000 di-IPTS ratios were poured in a 25 ml vial (30 mm diameter) and heated at 37° C. Then 1 ml PBS (pH 7.4 0.1 M) were added onto the material. The system was incubated at 37° C.

PCL530 di-IPTS Crosslinked Scaffold within Pluronic F127 matrix

The synthesis of PCL530 di-I:PTS was described above. 0.8 g F127 were dissolved in 3.2 g PBS (pH=7.4, 0.1 M) at 4° C. Then, 1 g PCL530 di-IPTS were added and the mixture was homogenized and incubated at 37° C.

F127 di-IPTS/PCL530 di-IPTS Crosslinked Copolymer

The synthesis of F127 di-IPTS and PCL530 di-IPTS was described above. 0.8 g F127 di-IPTS were dissolved in 3.2 g PBS (pH=7.4, 0.1 M) at 4° C. Then, 1 g PCL530 di-IPTS were added and the mixture was homogenized and incubated at 37° C.

PTMG2000 di-IPTS Crosslinked Scaffold within Pluronic F127 matrix

The synthesis of PTMG2000 CL di-IPTS was described above. 0.8 g F127 were dissolved in 3.2 g PBS (pH=7.4, 0.1 M) at 4° C. Then, 1 g PTMG2000 di-IPTS were added and the mixture was homogenized and incubated at 37° C.

F127 di-IPTS/PTMG2000 CL di-IPTS crosslinked copolymer

The synthesis of F127 di-IPTS and PCL530 di-IPTS was described above. 0.8 g F127 di-IPTS were dissolved in 3.2 g PBS (pH=7.4, 0.1 M) at 4° C. Then, 1 g PTMG2000 CL di-IPTS were added and the mixture was homogenized and incubated at 37° C.

5. Composition of the Grafts:

Grafts were composed of the following ingredients, in different combinations:

1. 10 μl of BMC suspension (concentration 3×10⁸ cells/ml);

2. 4 mg of DBM (or MBM, or DTM);

3. 10 μl of polymeric material solution.

6. Transplantation into the Sub-Capsular Space of the Kidney

Anaesthetized rats or mice were used as recipients. A small cut was made in the renal capsule and the transplanted material was inserted using a concave spatula. The transplant consisted of BMC suspension mixed with DBM powder with or w/o the supplement of RTG polymeric material. As a control BMC mixed with polymeric material or RTG polymeric material alone were transferred under the kidney capsule. The skin was closed with stainless clips.

7. Implantation of a Mixture of BMC, DBM and Polymeric Materials into the Area of Local Damage in the Articular Cartilage of the Knee Joint

A standard artificial damage in the articular cartilage and subchondral bone in the rat knee joint was induced as described. Following anesthesia, the knee joint was accessed by a medial Para patellar incision, and the patella was temporarily displaced towards the side. A microfracture drilling (for a full thickness defect) of 1.5 mm in diameter and 2.0 mm in depth was made in the interchondylar region of the femur.

The defect was filled with mixture of DBM powder with BMC suspension, prepared as described above, supplemented or not supplemented with polymeric material. As a control BMC mixed with polymeric material or polymeric material alone were transferred into the damaged area. Patella was returned into its place and the incision was sutured with bioresorbable thread. The skin was closed with stainless clips.

8. Implantation of a Mixture of BMC DBM and Polymeric Materials into the Experimentally Created Calvarial Defect.

Lewis rats were anesthetized by intraperitoneal injection of Ketamine. An incision was performed in the frontal region of the rat cranium. The muscular flap was removed from the parietal bone area and a bony defect (6×6 mm²) was made lateral to the saggital suture using a dental burr. The defect was filled with mixture of DBM powder and BMC suspension, prepared as described above, supplemented or not supplemented with polymeric material. As a control BMC mixed with polymeric material or polymeric material alone were transferred into the damaged area. The skin was closed with stainless clips.

9. Histological Evaluation

The autopsied material was fixed in 4% neutral buffered formaldehyde, decalcified, passed through a series of ethanol grades and xylene, and then embedded in paraffin. Serial sections (5-7 microns thick) were obtained. One set of representative serial sections of each sample was stained with Hematoxylin & Eosin (H&E), and another one with Picroindigocarmin (PIC).

### Example 1

### Study on the Influence of Various Polymeric Materials on Osteogeneic Properties of BMC-DBM Composition Transplanted into the Sub-Capsular Space of the Kidney in Mice

In the following examples, the experimentation involved in the development of the composition of the invention. Several polymeric materials disposing high viscosity were found to be highly compatible with the process of induced bone development by mesenchymal stem cells persisting in the bone marrow (BM) transplanted together with DBM or DTM into the sub-capsular space of the kidney.

The space under the kidney capsule was selected as the site of transplantation, since it has been previously shown that it has no cells, which could be induced into osteogenesis and to build a bone, at least within the period of 2-3 months, thus being able to serve as an in vivo experimental tube for study the process of osteogenesis. [Gurevitch, O. A. et al. (1989) Hematol Transfusiol 34:43-45 (in Russian)].

In several sets of experiments carried out in rats and mice various site-responsive polymeric materials were studied in transplantation under the kidney capsule for their fitness to criterions mentioned above. The transplant consisted of BMC suspension mixed with DBM or DTM powder with or w/o the supplement of either RTG or silane-based polymeric materials. As a control BMC mixed with polymeric material or polymeric material alone were transferred into the sub-capsular space of the kidney. No less than 5 transplantations were performed per group.

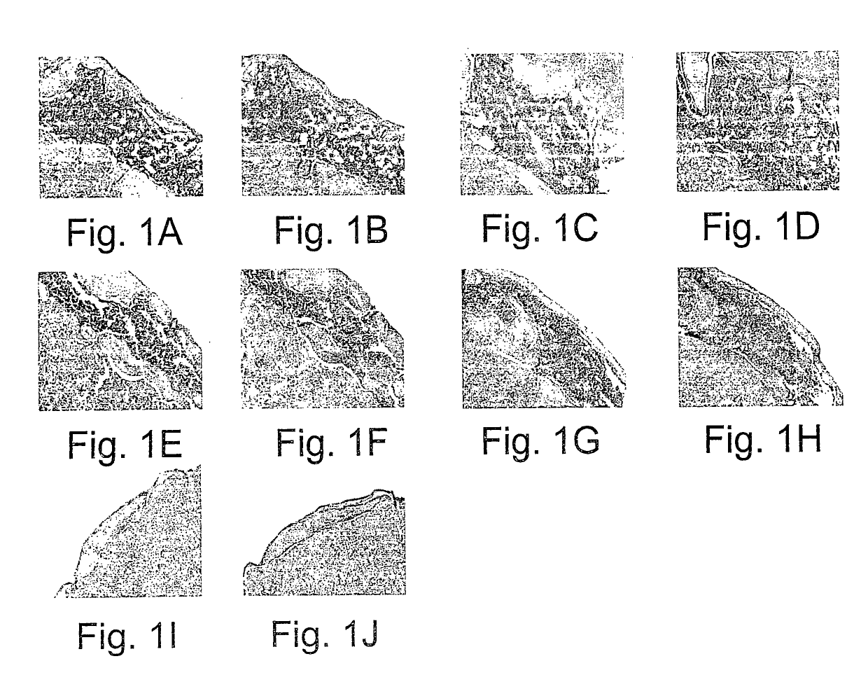

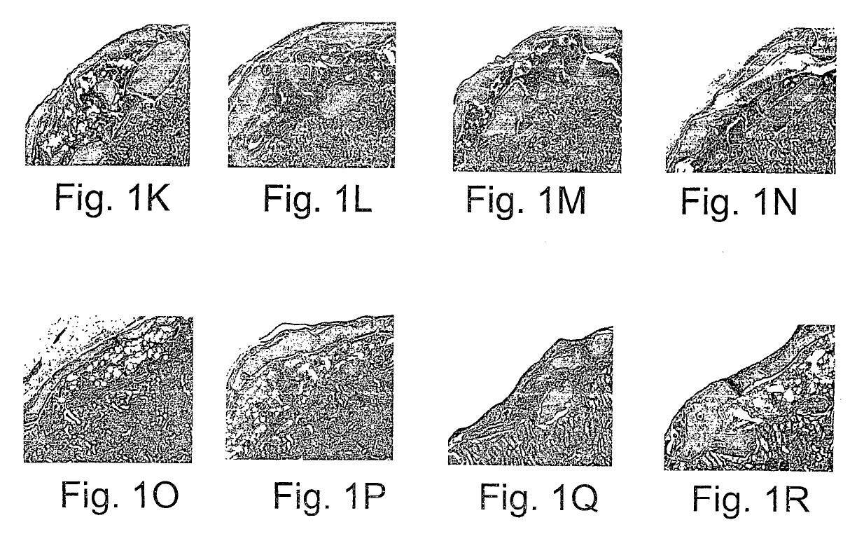

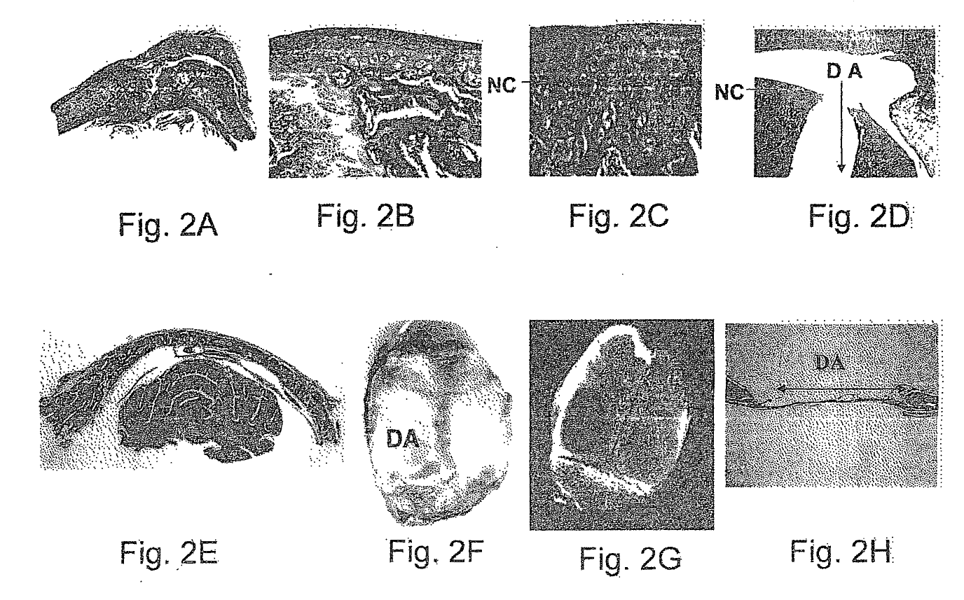

One month post-transplantation of BMC+DTM together with RTG polymers (N2, N4, N7) newly formed cortical and trabecular bone, well developed marrow cavity and functionally active bone marrow are seen in all the cases (FIG. 1A-J).

FIGS. 1K to 1R show the influence of modified Pluronic F-127 RTG biopolymers on the development of osteohematopoietic site induced by transplantation of the mixture containing BMC+DTM/DBM+biopolymers #21 (FIGS. 1L and 1P), #22 (FIGS. 1M and 1Q) and #23 (FIGS. 1N and 1R). The transplantation site was either mouse (FIG. 1K-1N) or rat kidney capsule (FIG. 1O-1R).

One month post-transplantation of BMC+DBM (in rats) or BMC+DTM (in mice) together with the site-responsive polymers #21, #22 and #23 induced de novo formation of cortical and trabecular bone, with an well developed marrow cavity and a functionally active bone marrow (FIG. 1K-1R). No difference in the developmental level of ectopic ossicles produced by DTM-BMC complex transplanted with or without RTG polymers could be observed. These three polymers correspond to the concentrations of 15, 17 and 20% of polymer, respectively, in water. None of these concentrations display any adverse effect. It is important to note that these high concentrations of polymer are beneficial for improved mechanical properties of the graft.

BMC transplanted without DBM/DTM but supplemented with each of the mentioned above RTG polymers produced in most of the cases small ossicles as it used to be when transplanted BMC, which here are kept together and their migration out of the transplantation site is prevented (FIG. 1). It seems most probable that in the absence of osteo-inductive and osteo-conductive influences of DBM mesenchymal stem cells could not be effectively induced into osteogenesis, thus only predifferentiated (restricted to osteo-chondrogenesis) progenitor cells existing in the transplanted BMC are engaged in osteogenesis.

Implantation of the above mentioned polymeric materials alone under the kidney capsule never left any trace in the site of transplantation—neither bone formation nor any side effects such as inflammation etc.

These experiments show that the osteo- and chondrogenic processes are not bound by the different concentrations of the site-responsive polymer, at least within the range applied herein. This can be advantageous during the transplantation procedure, wherein the concentration of choice can be dependent on the time constraints of the implantation procedure. Thus, in cases where a more extended period of time for formation of the depot is recommended or desired, it would be convenient to use a mixture with lower site-responsive polymer concentrations, which will take longer to form a depot at the site. In counterpart, when less time is desired for the polymerization to take place, the mixture with higher polymer concentration shall be used.

### Example 2

### Study on the Influence of Various RTG Polymeric Materials on Correction of Experimentally Created Calvarial Defect Induced by Transplantation of BMC-DBM Composition

Experiments were carried out to test whether the polymeric materials that were chosen in the previous set of experiments for their viscosity and high compatibility with the process of induced bone development by mesenchymal stem cells of BM transplanted together with DBM are able to improve the correction of the experimentally created calvarial defect.

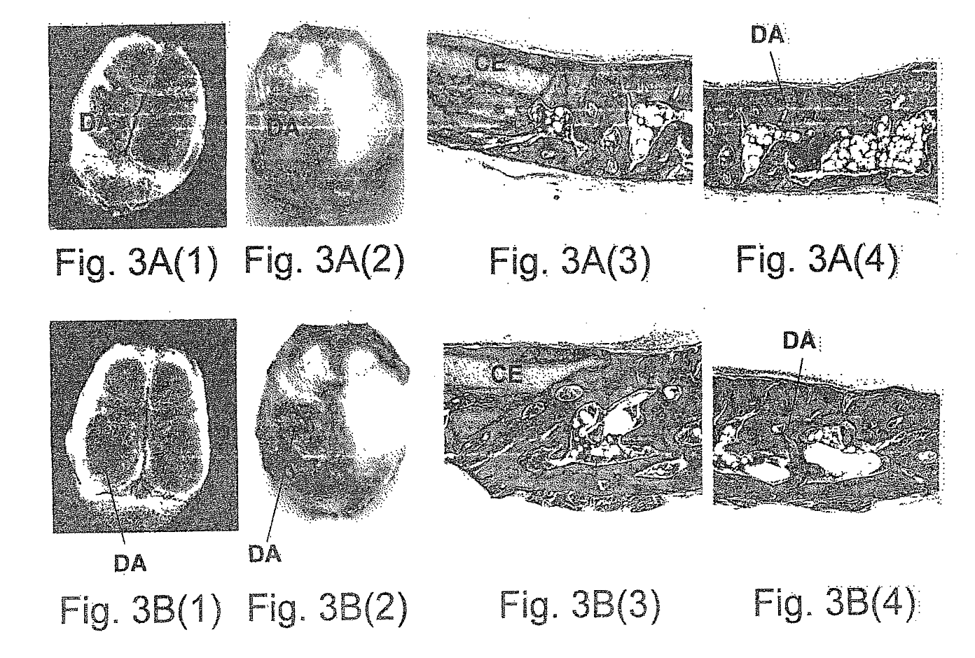

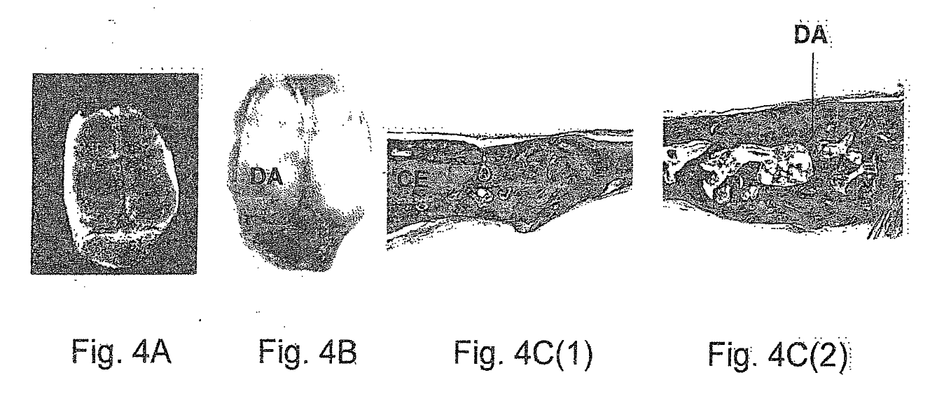

It was shown that transplantation of composition of the invention comprised of BMC, DBM and each of the chosen RTG polymeric materials could initiate and accomplish the intramembranous development of bone, when transplanted into the experimentally created calvarial defect. The results of these experiments are shown in FIGS. 3-5. This method could then be extended to treat facial-maxillary defects.

An incision was performed in the frontal cranium region of anesthetized Lewis rats (8-12 weeks old) and the skin flap was moved aside. The muscular flap was removed from the parietal bone area and a bony defect was created laterally to the sagital suture using a dental burr, full width segment of parietal bone (6×6 mm²) was removed. The defect area was filled with BMC suspension mixed with DBM powder with or w/o the supplement of RTG polymeric material. As a control BMC mixed with polymeric material or polymeric material alone were transferred into the experimentally created calvarial defect. The skin flap was returned to place and fixed with stainless clips. No less than 5 transplantations were performed per group.

The utilization of non-healing cranial defects allows for the observation of both osteo-conductive and osteo-inductive components of the healing process. Thus, the non-healing cranial defect represents an appropriate model for evaluating the ability of the composition of the present invention to accomplish intramembranous bone formation when transplanted into a damaged area of the crania.

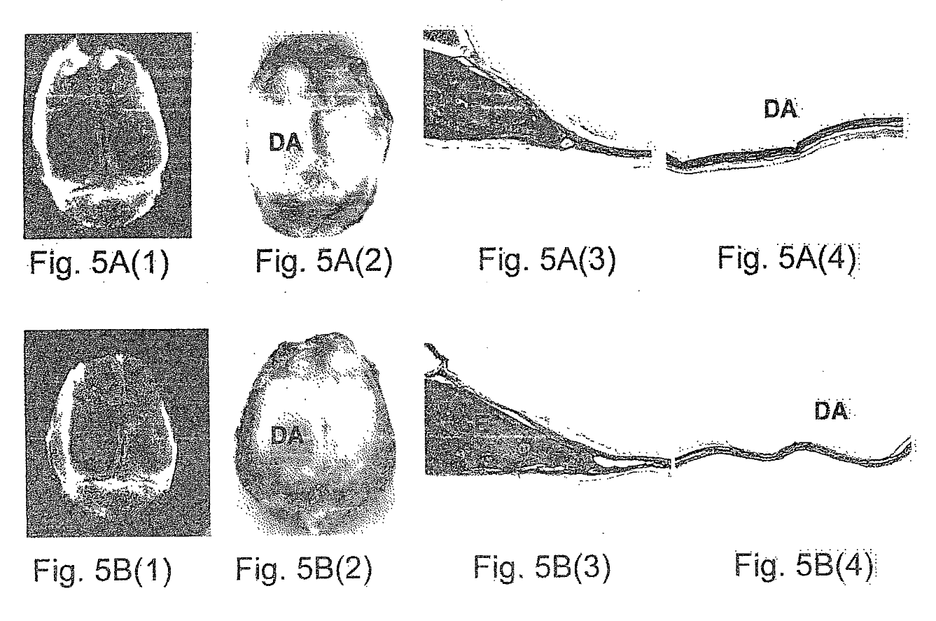

When the site of removed bone was filled with BMC together with each of the investigated RTG polymeric materials, no bone regeneration could be observed 30 days after the operation. It could be clearly seen in x-Ray and Macro pictures and confirmed by histological studies (FIG. 5), suggesting that the size of the defect sufficiently large, compatible with the definition of non-healing cranial defect.

Filling of the experimental cranial defect with each of the polymeric materials alone never left any trace in the site of transplantation—neither bone formation nor any side effects such as inflammation etc.

When BMC-DBM active composition supplemented with one of the mentioned above RTG polymeric materials was transplanted into the site of the experimental cranial defect, extensive remodeling of the transplanted DBM particles and developing areas of new bone could be observed. As early as one month after transplantation the cut edge of the parietal bone could hardly be distinguished from the surrounding new bony tissue. The defect area was fully reconstituted with a continuous layer of newly developing bone which could be clearly seen in x-Ray and Macro pictures and confirmed by histological studies (FIGS. 3 & 4)

It should be especially stressed that extensive remodeling of transplanted DBM particles and active new bone formation were presented evenly throughout the defect area, suggesting that the quantity of available active complex consisting of BMC (containing mesenchymal progenitor cells capable of being induced and conducted to osteogenesis) and DBM particles was maintained uniformly in the defect area.

These findings indicate that supplementation of the active complex composed of BMC and DBM with said RTG polymeric materials i.e. usage the composition of present invention (in this case, DBM together with BMC and said polymeric materials) for transplantation into an experimentally created calvarial defect was sufficient for preventing the transplant from disintegration, keeping the transplant in the proper site preserving its shape, providing active and complete intramembranous bone formation at the site of transplantation. This procedure could be extended to treat facial-maxillary defects.

It has to be especially emphasized that without the application of polymeric materials transplantation of BMC-DBM complex into an experimentally created calvarial defect resulted in non-uniform bone formation suggesting partial disintegration of the transplant and impossibility of keeping its initial shape.

Pilot experiments utilizing BMC in combination with MBM rather than DBM showed positive results. Mainly, the difference between employing DBM and MBM lies on delayed bone formation with MBM. Also, since MBM particles are much more dense and hard, as compared to DBM particles, they are more useful when weight bearing or shape preservation of the transplant are needed. Transplantation of a mixture of both DBM and MBM together with BMC should enable the best of the advantages of both: (a) significantly prolonging the period of osteogenic activity (with DBM acting fast and MBM after a delay); (b) improving the shape preservation of the implant throughout the whole period of new tissue formation.

### Example 3

### Study on the Influence of Various RTG Polymeric Materials on Osteogeneic Properties of BMC-DBM Composition Transplanted into the Area of Local Damage in the Articular Cartilage of the Knee Joint

Experiments were carried out to test whether the polymeric materials that were chosen in the previous set of experiments for their viscosity and high compatibility with the process of induced bone development by mesenchymal stem cells of BM transplanted together with DBM are able to improve the correction of the experimentally damaged osteochondral complex of the knee joint.

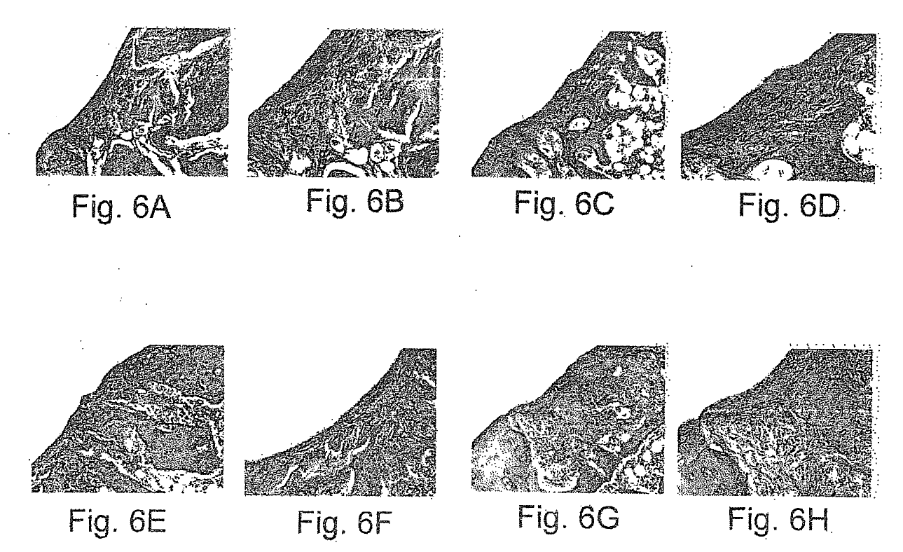

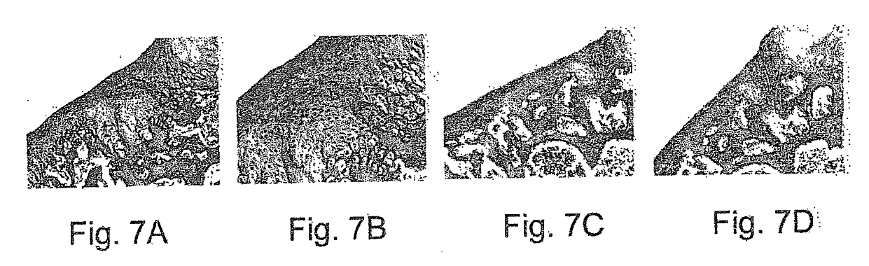

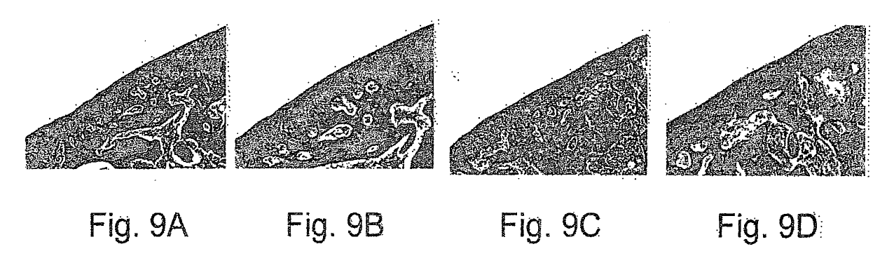

It was shown that transplantation of composition of the invention comprised of BMC, DBM and some of the chosen RTG polymeric materials could initiate and accomplish the process bone and cartilage development, when transplanted into the experimentally damaged osteochondral complex of the knee joint. The results of these experiments are shown in FIGS. 6-9. This method could then be extended to treat defects in osteochondral complex of the joints.

Male Lewis rats were anesthetized by intraperitoneal injection of Ketamine. Microfracture drilling (full thickness defect) was inflicted in articular cartilage and subchondral bone in the interchondylar region of the femur. The defect area was filled with BMC suspension mixed with DBM powder with or w/o the supplement of RTG polymeric material. As a control BMC mixed with polymeric material or polymeric material alone were transferred into the experimentally created damaged areas of the knee joints. Patella was returned into its place and the incision was sutured with bioresorbable thread. The skin was closed with stainless clips.

No less than 5 transplantations were performed per group.

One month after the site of osteo-chondral defect in the knee joint was filled with DBM-BMC complex together with each of the investigated RTG polymeric materials active regeneration of subchondral bone and hematopoietic cavities, angiogenesis as well as partial degradation and remodeling of DBM particles are seen.

However, the dramatic difference was observed in the way of regeneration of the damaged surface area when different polymeric materials were added. When DBM-BMC complex was transplanted into the damaged area accompanied by RTG polymer N7 regenerating surface was built of thick layer of young hyaline cartilage. On the contrary, when DBM-BMC active complex was transplanted with the supplement of RTG polymers N2 or N4 no cartilage formation was observed, regenerating surface was built of connective tissue.

When BMC together with mentioned above RTG polymeric materials were transplanted into the damaged osteo-chondral complex of the knee joint regeneration followed the same general pattern. Active regeneration of subchondral bone and hematopoietic cavities was seen with all the polymeric materials. However regenerating surface was built of connective tissue alone when BMC were accompanied by RTG polymers N2 or N4, while in the cases in which BMC were supplemented with RTG N7 regenerating surface of the damaged area comprised a mixture of connective tissue with cartilage cells.

Interestingly, RTG polymeric materials N2 and N4 proved to selectively prevent the process of chondrogenesis induced by transplantation of DBM-BMC active complex into the damaged osteochondral area of the knee joint while, being compatible with the process of induced osteogenesis in the same site.

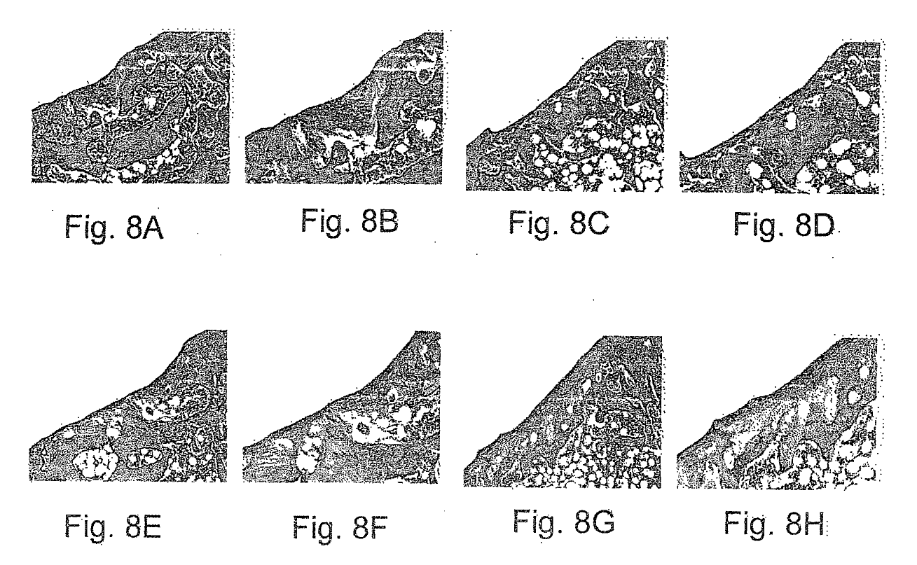

Two months observation of regeneration patterns of damaged osteochondral complex after DBM-BMC active composition was transplanted supplemented with different RTG polymeric materials completely confirmed the results obtained in one month (FIGS. 8 & 9).

It should be stressed that transplantation of the composition of invention (in this case BMC and DBM together with said polymeric materials) into experimentally performed full thickness damage in the osteo-chondral complex of the knee joint allowed to maintain smooth and uniform regenerating surface in the defect area which is especially important for complete rehabilitation of the joint.

These findings indicate that using composition of the present invention (in this case, DBM together with BMC and said RTG polymeric materials) for transplantation into an experimentally created defect in osteo-chondral complex of the knee joint was sufficient for preventing the transplant from disintegration, keeping the transplant in the proper site preventing the DBM particles from thrusting out of the transplantation site into the articular surface, providing formation of fully developed osteochondral complex and the smooth regenerating surface of hyaline cartilage. This procedure could be extended to treat osteo-chondral defects in the joints.

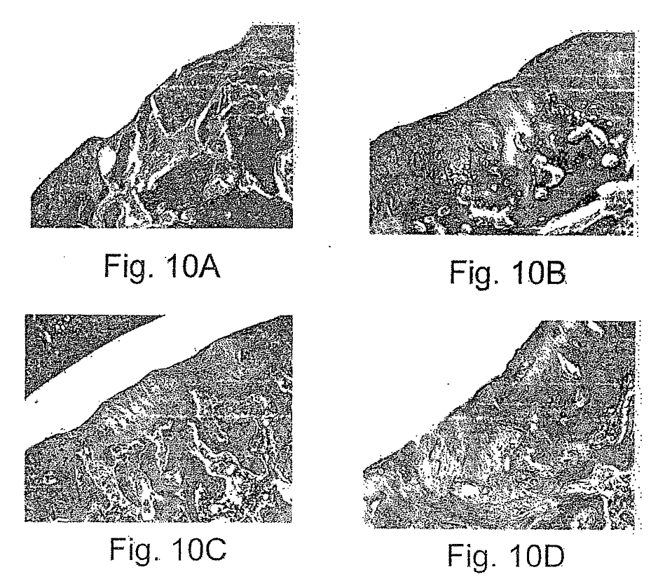

It should be pointed out that without the supplementation with polymeric materials transplantation of BMC-DBM complex into an experimentally created defect in osteo-chondral complex of the knee joint resulted in formation of non-uniform regenerating surface as the result of partial disintegration of the transplant and thrusting of DBM particles out of the site of transplantation into the articular surface of the joint (FIG. 10).

1-50. (canceled)

51. A combination of:
at least one polymer in an aqueous solution which is capable of undergoing a transition that results in an at least two-fold increase in its viscosity, said at least one polymer being characterized by at least one of the following features:
having a viscosity at body temperature which is higher than 22,000 Pas following said transition;
having a molecular weight higher than 17,000 Daltons;
comprising a block copolymer having more than three blocks;
having a polymer degradation time generally similar to the time required for de novo chondrogenisis and osteogenesis; and
capable of undergoing a condensation reaction in the presence of water resulting in an increase in the molecular weight of the polymeric system; and
at least one of a source of Mesenchymal stem cells and at least one of demineralized bone matrix, demineralized tooth matrix, bone morphogenic protein and bone morphogenic growth factor.

52. A combination according to claim 51 and wherein said combination includes both said source of Mesenchymal stem cells and said at least one of demineralized bone matrix, demineralized tooth matrix, bone morphogenic protein and bone morphogenic growth factor.

53. A combination according to claim 51 and wherein said combination includes said source of Mesenchymal stem cells and does not include said at least one of demineralized bone matrix, demineralized tooth matrix, bone morphogenic protein and bone morphogenic growth factor.

54. A combination according to claim 51 and wherein said combination includes said at least one of demineralized bone matrix, demineralized tooth matrix, bone morphogenic protein and bone morphogenic growth factor and does not include said source of Mesenchymal stem cells.

55. A combination according to claim 51 and wherein said at least one polymer in an aqueous solution is capable of undergoing said transition in response to a triggering effect at a body site.

56. A combination according to claim 55 and wherein said at least one polymer in an aqueous solution is capable of undergoing said transition in response to said triggering effect which includes a change in at least one of temperature and PH.

57. A combination according to claim 52 and wherein said at least one polymer in an aqueous solution is capable of undergoing said transition in response to a triggering effect at a body site.

58. A combination according to claim 57 and wherein said at least one polymer in an aqueous solution is capable of undergoing said transition in response to said triggering effect which includes a change in at least one of temperature and PH.

59. A combination according to claim 51 and wherein said at least one polymer in an aqueous solution is characterized by at least one of the following features:
having a molecular weight higher than 17,000 Daltons;
comprising a block copolymer having more than three blocks; and
having a polymer degradation time generally similar to the time required for de novo chondrogenisis and osteogenesis.

60. A combination according to claim 52 and wherein said at least one polymer in an aqueous solution is characterized by at least one of the following features:
having a molecular weight higher than 17,000 Daltons;
comprising a block copolymer having more than three blocks; and
having a polymer degradation time generally similar to the time required for de novo chondrogenisis and osteogenesis.

61. A combination according to claim 51 and wherein said at least one polymer in an aqueous solution is characterized by at least one of the following features:
having a molecular weight higher than 17,000 Daltons;
comprising a block copolymer having more than three blocks; and
having a viscosity at body temperature which is higher than 22,000 Pas following said transition.

62. A combination according claim 51 and wherein said at least one polymer in an aqueous solution is characterized by at least one of the following features:
having a molecular weight higher than 17,000 Daltons; and
comprising a block copolymer having more than three blocks.

63. A combination according to claim 51 and wherein said at least one polymer in an aqueous solution is characterized in having a viscosity at body temperature which is higher than 22,000 Pas following said transition.

64. A combination according to claim 51 and wherein said source of Mesenchymal stem cells is bone marrow.

65. A combination according to claim 51 and wherein said combination includes at least one of said demineralized bone matrix and said demineralized tooth matrix.

66. A combination according to claim 51 and wherein said combination also comprises living cells selected from the group consisting of endothelial cells, hepatocytes, astrocytes, osteoblasts, chondrocytes, fibroblasts and miocytes.

67. A combination according to claim 51 and wherein:
said combination includes said source of Mesenchymal stem cells;
said source of Mesenchymal stem cells is bone marrow;
said combination also includes said demineralized bone matrix;
said at least one polymer in an aqueous solution is a segmented block copolymer exhibiting reverse thermo gelating properties and comprising polyethylene oxide (PEO) and polypropylene oxide (PPO) chains;
said chains are connected via a chain extender; and
said chain extender is phosgene.

68. A method for regenerating musculoskeletal tissue other than bone, the method comprising:
forming a combination of at least one polymer in an aqueous solution which is capable of undergoing a transition that results in an at least two fold increase in its viscosity and a source of Mesenchymal stem cells; and
thereafter applying the combination to existing musculoskeletal tissue.

69. A method according to claim 68 and further comprising combining at least one of demineralized bone matrix, demineralized tooth matrix, bone morphogenic protein and bone morphogenic growth factor with said combination prior to said applying.

70. A method according to claim 69 and wherein said transition takes place in response to a triggering effect at a body site.

71. A method according to claim 70 and wherein said transition takes place in response to said triggering effect which includes a change in at least one of temperature and PH.

72. A method according to claim 69 wherein said source of Mesenchymal stem cells is bone marrow.

73. A method according to claim 69 and wherein said at least one of demineralized bone matrix, demineralized tooth matrix, bone morphogenic protein and bone morphogenic growth factor includes at least one of said demineralized bone matrix and said demineralized tooth matrix.

74. A method according to claim 69 and also comprising combining living cells selected from the group consisting of endothelial cells, hepatocytes, astrocytes, osteoblasts, chondrocytes, fibroblasts and miocytes with said combination prior to said applying.

75. A method for regenerating musculoskeletal tissue comprising:
initially forming a combination of a source of Mesenchymal stem cells and at least one of demineralized bone matrix, demineralized tooth matrix, bone morphogenic protein and bone morphogenic growth factor;
thereafter adding to said combination at least one polymer in an aqueous solution which is capable of undergoing a transition that results in an at least two fold increase in its viscosity, and;
thereafter applying the combination to existing musculoskeletal tissue.

76. A method according to claim 75 and wherein said transition takes place in response to a triggering effect at a body site.

77. A method according to claim 76 and wherein said transition takes place in response to said triggering effect which includes a change in at least one of temperature and PH.

78. A method according to claim 75 wherein said source of Mesenchymal stem cells is bone marrow.

79. A method according to claim 75 and wherein said at least one of demineralized bone matrix, demineralized tooth matrix, bone morphogenic protein and bone morphogenic growth factor includes at least one of said demineralized bone matrix and said demineralized tooth matrix.

80. A method according to claim 75 and also comprising combining living cells selected from the group consisting of endothelial cells, hepatocytes, astrocytes, osteoblasts, chondrocytes, fibroblasts and miocytes with said combination prior to said applying.
